(19) United States
(12) Patent Application Publication
(10) Pub. No.: US 2022/0167894 A1
(43) Pub. Date: Jun. 2, 2022

(54) PHYSIOLOGICAL STATE CONTROL APPARATUS, PHYSIOLOGICAL STATE CHARACTERISTIC DISPLAY APPARATUS, AND PHYSIOLOGICAL STATE CONTROL METHOD

(71) Applicant: NEC Corporation, Minato-ku, Tokyo (JP)

(72) Inventor: Takuma KOGO, Tokyo (JP)

(73) Assignee: NEC Corporation, Minato-ku, Tokyo (JP)

(21) Appl. No.: 17/601,752

(22) PCT Filed: Feb. 13, 2020
(86) PCT No.: PCT/JP2020/005454

§ 371 Date: Oct. 6, 2021

(30) Foreign Application Priority Data
Apr. 10, 2019 (JP) 2019-075056

(51) Int. Cl. A61B 5/16; A61B 5/00

(52) U.S. Cl. CPC A61B 5/16; A61B 5/7264

(57) ABSTRACT

A physiological state control apparatus includes mixing ratio computation means for computing, on the basis of characteristic data regarding a subject, mixing ratios for multiple sub-models that take, as an input, a physical quantity in a space in which the subject is located and that output a predicted value of a physiological index; model generation means for generating a physiological state prediction model for the subject on the basis of the mixing ratios and the sub-models; and device control means for controlling a control target device that influences the physical quantity using the physiological state prediction model.

### TECHNICAL FIELD

The present invention relates to a physiological state control apparatus, a physiological state characteristic display apparatus, a physiological state control method, a physiological state characteristic display method, and a computer-readable recording medium storing a program.

### BACKGROUND ART

Technologies for acquiring biological information from a user and computing the arousal level of the user from the acquired biological information have been proposed (e.g., Patent Documents 1 and 2). Here, an arousal level is an index for indicating the degree to which a subject is awake. A lower arousal level value indicates that the subject is in a drowsy state.

In a low arousal level state, the work efficiency is often lowered when the user is performing work, and the user is not in a state that is suitable for carrying out the work. There is a tendency to be in an undesired state for each kind of work; for example, in office work, the work efficiency becomes lower, and when driving an automobile, distracted driving increases.

For this reason, systems that control the environment around a user so that an arousal level is increased or the arousal level is within an appropriate range have been proposed (Patent Documents 3, 4 and 5).

Patent Document 3 discloses a system for controlling an arousal level, for drivers of vehicles, wherein the settings of devices for controlling environments, such as air conditioning and lighting, are changed to predetermined settings when a predicted value of the arousal level of a user becomes lower than a predetermined threshold value in the case in which the current environmental state is maintained.

Patent Document 4 discloses a system for controlling an arousal level, for drivers of vehicles, wherein a combination of devices stimulating the five senses, such as an air conditioning device and a lighting device, and the intensity levels of air conditioning and lighting are determined on the basis of predetermined settings, depending on where the user's current state is located, particularly how far the user's current state is located outside a desired range, in terms of biaxial coordinates consisting of a drowsiness-arousal level evaluation axis and a comfort-discomfort evaluation axis, and these devices are controlled on the basis of the determined combination of the devices and the determined intensity levels.

Patent Document 5 discloses a system for controlling an arousal level, for drivers of vehicles, wherein a user is subjected to hot/cold stimulation due to temperature changes by periodically switching between predetermined operating modes (temperature and air volume settings) of an air conditioning device when the arousal level of a subject has become below a predetermined threshold value.

Additionally, there are technologies that acquire information on a user or information on a surrounding environment around the user and perform processes.

For example, in a mood estimation system in Patent Document 6, the mood of a subject is indexed on the basis of only the heart rate of the subject, and if the index value goes outside a predetermined range, then the mood of the subject is indexed on the basis of multiple types of biological information regarding the subject and multiple types of environmental information regarding a surrounding environment around the subject.

Additionally, an air conditioning management system described in Patent Document 7 computes a predicted environmental value for a predetermined time in the future on the basis of an environmental value detected by a detection apparatus, computes parameters for an air conditioning apparatus on the basis of the environmental value and the predicted environmental value, and transmits the computed parameters to the air conditioning apparatus.

Additionally, in an arousal level maintenance method described in Patent Document 8, an arousal level is detected from a core body temperature, such as the tympanic temperature, of a worker, and when a drop in the arousal level of the worker is observed, the illuminance is changed from an illuminance suitable for working to a higher illuminance, thereby providing arousal effects based on stimulation with light to the worker.

Additionally, a drowsiness estimation apparatus described in Patent Document 9 is provided with a neural network having a two-layered structure consisting of an image-processing neural network and a drowsiness-estimating neural network. The image-processing neural network estimates the age and gender of the user, and extracts specific actions and states of the user indicating a drowsy state, such as the eyes being closed. The drowsiness-estimating neural network considers the user's age and gender to determine the drowsiness state of the user on the basis of the results of extraction of the specific actions and states of the user indicating a drowsy state, and the results of detection by an indoor environmental information sensor.

This Patent Document 9 describes that a control unit in an air conditioning apparatus computes air conditioning control content for lowering the estimated drowsiness level to a threshold value or lower, and executes air conditioning control as indicated by the computed air conditioning control content. Furthermore, Patent Document 9 describes that an estimated model is updated if a desired change is not observed in the actions and state of the user because there is a possibility that the actions for estimating a drowsy state are departing from an actual drowsy state.

### PRIOR ART DOCUMENTS

### Patent Documents

Patent Document 1: Japanese Patent No. 6043933
Patent Document 2: Japanese Unexamined Patent Application, First Publication No. 2018-134274
Patent Document 3: Japanese Unexamined Patent Application, First Publication No. 2017-148604
Patent Document 4: Japanese Unexamined Patent Application, First Publication No. 2018-025870
Patent Document 5: Japanese Unexamined Patent Application, First Publication No. 2013-012029
Patent Document 6: Japanese Unexamined Patent Application, First Publication No. 2018-088966
Patent Document 7: Japanese Unexamined Patent Application, First Publication No.

2006-349288
Patent Document 8: Japanese Unexamined Patent Application, First Publication No. H09-140799
Patent Document 9: Japanese Patent No. 6387173

### SUMMARY

### Problem to be Solved by the Invention

When an apparatus or a system controls a physiological state by acting on a surrounding environment around a subject of physiological state control, such as arousal level control, there are individual differences and differences due to the subject's psychosomatic state in the degree of influence that the surrounding environment has on the subject. In order to control the physiological state with high precision, the physiological state control should preferably reflect the individual differences and differences due to the subject's psychosomatic state in the degree of influence that the surrounding environment has on the subject.

An example object of the present invention is to provide a physiological state control apparatus, a physiological state characteristic display apparatus, a physiological state control method, a physiological state characteristic display method, and a computer-readable recording medium storing a program, which can solve the above-mentioned problem.

### Means for Solving the Problem

According to a first example aspect of the present invention, a physiological state control apparatus includes: mixing ratio computation means for computing, on the basis of characteristic data regarding a subject, mixing ratios for multiple sub-models that take, as an input, a physical quantity in a space in which the subject is located and that output a predicted value of a physiological index; physiological state prediction model generation means for generating a physiological state prediction model for the subject on the basis of the mixing ratios and the sub-models; and device control means for controlling a control target device that influences the physical quantity using the physiological state prediction model.

According to a second example aspect of the present invention, a physiological state characteristic display apparatus includes: mixing ratio computation means for computing, on the basis of characteristic data regarding a subject, mixing ratios for multiple sub-models that take, as an input, a physical quantity in a space in which the subject is located and that output a predicted value of a physiological index; and display means for displaying a degree of influence of the physical quantity on increases and decreases in a physiological index value for the sub-models and displaying the mixing ratios for each subject.

According to a third example aspect of the present invention, a physiological state control method performed by a computer includes: computing, on the basis of characteristic data regarding a subject, mixing ratios for multiple sub-models that take, as an input, a physical quantity in a space in which the subject is located and that output a predicted value of a physiological index; generating a physiological state prediction model for the subject on the basis of the mixing ratios and the sub-models; and controlling a control target device that influences the physical quantity using the physiological state prediction model.

According to a fourth example aspect of the present invention, a physiological state characteristic display method performed by a computer includes: computing, on the basis of characteristic data regarding a subject, mixing ratios for multiple sub-models that take, as an input, a physical quantity in a space in which the subject is located and that output a predicted value of a physiological index; displaying a degree of influence of the physical quantity on increases and decreases in a physiological index value for the sub-models; and displaying the mixing ratios for each subject.

According to a fifth example aspect of the present invention, a computer-readable recording medium stores a program for making a computer execute: a step of computing, on the basis of characteristic data regarding a subject, mixing ratios for multiple sub-models that take, as an input, a physical quantity in a space in which the subject is located and that output a predicted value of a physiological index; a step of generating a physiological state prediction model for the subject on the basis of the mixing ratios and the sub-models; and a step of controlling a control target device that influences the physical quantity using the physiological state prediction model.

According to a sixth example aspect of the present invention, a computer-readable recording medium stores a program for making a computer execute: a step of computing, on the basis of characteristic data regarding a subject, mixing ratios for multiple sub-models that take, as an input, a physical quantity in a space in which the subject is located and that output a predicted value of a physiological index; and a step of displaying a degree of influence of the physical quantity on increases and decreases in a physiological index value for the sub-models and displaying the mixing ratios for each subject.

### Example Advantageous Effects of the Invention

According to the present invention, physiological state control can be made to reflect at least one of individual differences and differences due to the psychosomatic state in the degree of influence that a physical quantity in a surrounding environment has on a subject of physiological state control.

### BRIEF DESCRIPTION OF DRAWINGS

FIG. 1 is a schematic block diagram illustrating an example of an apparatus configuration for an arousal level control system according to an example embodiment.

FIG. 2 is a schematic block diagram illustrating an example of a functional configuration of an arousal level control apparatus according to an example embodiment.

FIG. 3 is a flow chart indicating an example of a procedure for a process by which a setting value computation unit according to an example embodiment computes device setting values and sets them in environmental control devices.

FIG. 4 is a diagram illustrating an example of a procedure for a process by which the arousal level control apparatus according to an example embodiment generates an arousal level prediction model.

FIG. 5 is a diagram illustrating an example of a display of an input coefficient matrix by a display unit according to an example embodiment.

FIG. 6 is a diagram illustrating an example of a display of sub-model mixing ratio vectors by the display unit according to an example embodiment.

FIG. 7 is a diagram illustrating an example of a configuration of an arousal level control apparatus according to an example embodiment.

FIG. 8 is a diagram illustrating an example of a configuration of an arousal level characteristic display apparatus according to an example embodiment.

FIG. 9 is a diagram illustrating an example of a procedure for a process in an arousal level control method according to an example embodiment.

FIG. 10 is a diagram illustrating an example of a procedure for a process in an arousal level characteristic display method according to an example embodiment.

### EXAMPLE EMBODIMENT

Hereinafter, example embodiments of the present invention will be explained, but the example embodiments below do not limit the invention according to the claims. Additionally, not all combinations of the features explained in the example embodiments are necessarily essential to the solving means provided by the invention.

Additionally, hereinafter, an example of a case in which a physiological state control apparatus according to an example embodiment is configured as an arousal level control apparatus and control is performed so as to increase the arousal level of a subject of physiological state control (control of a physiological state) (e.g., so as to maximize the sum of the arousal levels of subjects of physiological state control) will be explained.

However, the physiological state to be controlled by the physiological state control apparatus according to the example embodiment is not limited to an arousal level. The physiological state mentioned here is a physical state, a mental state, or a state that is both physical and mental. The physiological state control apparatus according to the example embodiment controls the physiological state by controlling a physical quantity in a surrounding environment around a subject of physiological state control. In other words, the physiological state control apparatus according to the example embodiment has, among the physiological states, a physiological state, the degree of which can be represented by a numerical value, and the degree of which can be controlled by controlling the physical quantity in the surrounding environment around the subject of physiological state control, as a control target.

Here, the physical quantity in the surrounding environment around the subject is a physical quantity (a quantity that is physical) that has an influence on the subject, and particularly here, is a physical quantity that has an influence on the physiological state of the subject. The physical quantity in the surrounding environment around the subject will also be referred to simply as a physical quantity.

Additionally, an index indicating the degree of a physiological state will be referred to as a physiological index, and the value of a physiological index will be referred to as a physiological index value.

For example, the physiological state control apparatus according to the example embodiment may be configured as a fatigue level control apparatus and may perform control to decrease the fatigue level of a subject of physiological state control. Additionally, the physiological state control apparatus according to the example embodiment may be configured as a stress control apparatus and may perform control to decrease the stress of a subject of physiological state control. Additionally, the physiological state control apparatus according to the example embodiment may be configured as a comfort level control apparatus and may perform control so as to increase the comfort level of a subject of physiological state control. Additionally, the physiological state control apparatus according to the example embodiment may be configured as a relaxation level control apparatus and may perform control so as to increase the relaxation level of a subject of physiological state control.

Additionally, in the case in which the physiological state control apparatus according to the example embodiment is for controlling an arousal level, drowsiness may be used as the physiological index instead of an arousal level, and control may be performed so as to decrease the drowsiness of a subject of physiological state control. Additionally, the physiological state control apparatus according to the example embodiment may be configured as a deep sleep level control apparatus and may perform control so as to increase the deep sleep level of a subject of physiological state control.

Hereinafter, arousal level control using an arousal level prediction model will be explained by referring to four forms of the arousal level prediction model. Additionally, hereinafter, after explaining arousal level control using the arousal level prediction model and providing explanations that are common to the four forms of the arousal level prediction model, the four forms of the arousal level prediction model will be explained, respectively, as a first example embodiment to a fourth example embodiment.

It should be noted that as explained above, the physiological state to be controlled by the physiological state control apparatus according to the example embodiment is not limited to an arousal level. The expression “arousal level” used below may be replaced with “physiological index”, and the expression “arousal level control” may be replaced with “physiological state control”.

Alternatively, the expression “arousal level” used below may be replaced with a physiological index other than an arousal level, and the expression “arousal level control” may be replaced with physiological state control other than arousal level control. Furthermore, if the purpose is to minimize the physiological index value, then the purpose of maximizing an arousal level by arousal level control is replaced therewith. For example, the expression “arousal level” may be replaced with fatigue level, the expression “arousal level control” may be replaced with “fatigue level control”, and an expression indicating that the arousal level is to be increased may be replaced with an expression indicating that the fatigue level is to be decreased.

### <Description of Arousal Level Control Using Arousal Level Prediction Model and Description Common to all Forms of Arousal Level Prediction Model>

### [Common Apparatus Configuration]

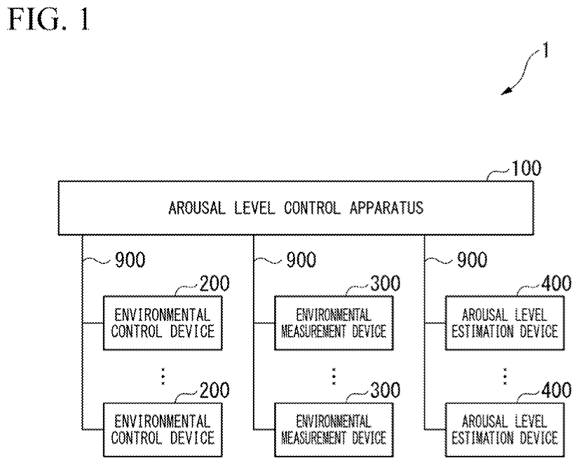

FIG. 1 is a schematic block diagram indicating an example of the apparatus configuration of an arousal level control system 1 according to an example embodiment. In the configuration indicated in FIG. 1, the arousal level control system 1 is provided with an arousal level control apparatus 100, one or more environmental control devices 200, one or more environmental measurement devices 300, and one or more arousal level estimation devices 400.

The arousal level control apparatus 100 is connected, via communication lines 900, to each of the environmental control devices 200, to each of the environmental measurement devices 300, and to each of the arousal level estimation devices 400, and is able to communicate with these devices. The communication lines 900 may be configured in any form, and the form thereof does not matter, including the form of exclusivity of the communication lines, such as whether they are dedicated lines, the internet, virtual private networks (VPNs), or local area networks (LANs), and the physical form of the communication lines, such as whether they are cable lines or wireless lines.

The arousal level control system 1 determines the arousal level of a subject of arousal level control and controls a physical quantity in a surrounding environment around the subject of arousal level control in accordance with the determination results to ensure that the arousal level is maintained or increased. As explained above, an arousal level is an index for indicating the degree to which a subject of arousal level control is awake. The lower the arousal level value is, the drowsier the subject of arousal level control is.

A subject of arousal level control will also be referred to as a user, a target user, or simply as a subject.

As mentioned above, the physical quantity in the surrounding environment around the subject mentioned here is a physical quantity that influences the physiological state of the subject. If the physiological state to be controlled is an arousal level, then the physical quantity in the surrounding environment around the subject is a physical quantity influencing the arousal level of the subject.

Examples of the physical quantity include air temperature, such as the room temperature, and brightness, such as the illuminance from a lighting device; however, the physical quantity is not limited thereto. For example, the arousal level control system 1 may, in addition to temperature and brightness, or instead of temperature and brightness, stimulate the subject with something other than temperature and brightness, such as moisture (humidity), sound, or vibrations, and may use the measures thereof as physical quantities.

The control of one of temperature, brightness, humidity, sound, and vibrations, or the control of combinations thereof, is expected to be effective even in the case that the physiological state to be controlled is fatigue level, stress level, comfort level, relaxation level, or deep sleep level. For example, the physiological state control apparatus or the physiological state control system according to the example embodiment may play music (make the subject hear music) and may use the sound volume at which the music is played as a physical quantity.

Hereinafter, the air temperature will be referred to simply as the temperature. However, the arousal level control system 1 may control the temperature of something else in addition to the air temperature or instead of the air temperature. The arousal level control system 1 may control the temperature of something directly contacting the subject; for example, a heater may be provided in a seat surface of the subject's seat and the arousal level control system 1 may control the temperature of the heater.

The units by which the arousal level control system 1 controls the physical quantity are not limited to specific units. For example, spot-type air conditioning devices (localized air conditioning devices) and lighting stands may be installed at the seats of individuals, and the arousal level control system 1 may control the physical quantity in units of seats. Alternatively, the arousal level control system 1 may control the physical quantity in units of rooms, or may control the physical quantity in an entire building. Additionally, in the case that the arousal level control system 1 controls the physical quantity in an entire building, the subjects do not need to be all of the people in the building, and may be just some of the people in the building.

The number of subjects may be one or more. The arousal level control system 1 may have only specific people as subjects, for example, accepting registration of the subjects. Alternatively, an unspecified person located in a control target space of the arousal level control system 1 may be a subject. In the case that there are multiple subjects, the arousal level control system 1 may control the physical quantity separately for each subject, or may control the physical quantity centrally for the multiple subjects.

In order to increase the arousal level of the subject, controlling the physical quantity so as to lower the comfort level for some people, for example, by raising the room temperature or by brightening the lighting, might be contemplated. By determining the arousal level of the subject of arousal level control and controlling the physical quantity in accordance with the determination results, the arousal level control system 1 can achieve a balance between comfort and ensuring the arousal level of the subject. For example, the arousal level control system 1 may control the physical quantity so as to increase the arousal level only when the arousal level of the subject has become low.

Hereinafter, the case in which the arousal level control system 1 increases the arousal level of (wakes up) a subject will be explained as an example; however, the arousal level control system 1 may decrease the arousal level of (induce sleep in) the subject. Furthermore, the arousal level control system 1 may increase the deep sleep level of (induce deep sleep in) the subject.

For example, the arousal level control system 1 may perform control so as to switch between control for increasing an arousal level and control for decreasing an arousal level in accordance with the hour of day. Alternatively, if the arousal level of the subject is expected to decrease, then the arousal level control system 1 may perform control so that the arousal level of the subject does not decrease (i.e., the subject does not become sleepy). Alternatively, if the arousal level of the subject is expected to increase, then the arousal level control system 1 may perform control so that the arousal level of the subject does not increase (i.e., the subject does not wake up).

The arousal level control apparatus 100 controls the environmental control devices 200 in accordance with the arousal level of the subject. The arousal level control apparatus 100 controls the physical quantities in the surrounding environment around the subject by controlling the environmental control devices 200, thereby controlling the arousal level of the subject.

The arousal level control apparatus 100 is formed, for example, by using a computer such as a personal computer (PC) or a workstation.

The environmental control devices 200 are devices that regulate the physical quantities. As explained above, the physical quantities may, for example, include the air temperature, the illuminance, and the like. The temperature can be regulated by means of an air conditioning device and the illuminance can be regulated by means of a lighting device. In this way, an air conditioning device and a lighting device can be mentioned as examples of the environmental control devices 200; however, the environmental control devices 200 are not limited thereto.

The environmental control devices 200 are examples of control target devices, and are controlled by the arousal level control apparatus 100 as described above.

Apparatuses other than the environmental control devices 200, such as the arousal level control apparatus 100, may acquire information relating to the operation state, such as device setting values, from the environmental control devices 200, and may update the device setting values of the environmental control devices 200. Here, the device setting values are physical quantities that are set in the environmental control devices 200 as control target values. The device setting values will also be referred to as physical quantity setting values or simply as setting values.

In the case that an environmental control device 200 is an air conditioning device, a set temperature may be used as a device setting value. In the case that an environmental control device 200 is a lighting device, a lighting output (e.g., light intensity, illuminance, an electric current value, an electric power value, etc.) may be used as a device setting value. Hereinafter, the case in which illuminance is used as the device setting value of a lighting device will be explained as an example; however, the device setting value of the lighting device is not limited thereto.

The environmental measurement devices 300 are devices that measure physical quantities such as temperature and illuminance and that convert the measured physical quantities to numerical data. A temperature sensor and an illuminance sensor can be mentioned as examples of the environmental measurement devices 300; however, the environmental measurement devices 300 are not limited thereto.

The arousal level estimation devices 400 are devices that estimate the arousal level of a subject from biological information or the like and that convert the estimated arousal level to numerical data. The arousal level estimation devices 400 may use any one of body temperature, video of the face, and pulse waves, or combinations thereof, as the biological information; however, the biological information is not limited thereto. The arousal level estimation devices 400 measure or compute the biological information and convert the obtained biological information to a numerical value (an arousal level) indicating the degree of arousal.

The arousal level estimation devices 400 mentioned here are an example of the case in which the physiological state to be controlled is an arousal level.

In the case in which the physiological state to be controlled is a physiological state other than an arousal level, the physiological state control system according to the example embodiment is provided with devices that can measure or compute physiological index values for the physiological state to be controlled instead of the arousal level estimation devices.

### [Common Functional Configuration]

Next, the functional configuration of the arousal level control apparatus 100 will be explained.

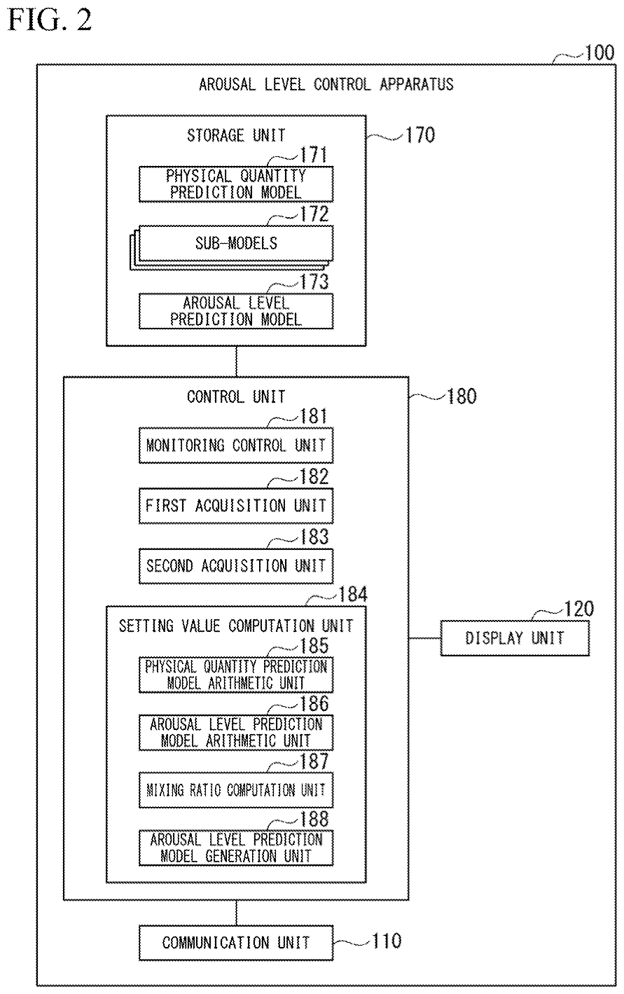

FIG. 2 is a schematic block diagram indicating an example of the functional configuration of the arousal level control apparatus 100. In the configuration shown in FIG. 2, the arousal level control apparatus 100 is provided with a communication unit 110, a display unit 120, a storage unit 170, and a control unit 180. The control unit 180 is provided with a monitoring control unit 181, a first acquisition unit 182, a second acquisition unit 183, and a setting value computation unit 184. The setting value computation unit 184 is provided with a physical quantity prediction model arithmetic unit 185, an arousal level prediction model arithmetic unit 186, a mixing ratio computation unit 187, and an arousal level prediction model generation unit 188 (arousal level prediction model generation means).

The communication unit 110 communicates with other apparatuses in accordance with control by the control unit 180. In particular, the communication unit 110 receives various types of information from each of the environmental control devices 200, each of the environmental measurement devices 300, and each of the arousal level estimation devices 400. Additionally, the communication unit 110 transmits device setting values to the environmental control devices 200.

The storage unit 170 stores various types of information. The storage unit 170 is configured by using a storage device provided in the arousal level control apparatus 100.

The storage unit 170 is provided with a physical quantity prediction model 171, sub-models 172, and an arousal level prediction model 173 generated by the arousal level prediction model generation unit 188.

The physical quantity prediction model 171 is a mathematical model for computing predicted values of physical quantities on the basis of setting values (device setting values) for those physical quantities.

More specifically, the physical quantity prediction model 171 computes predicted values of physical quantities for the time at which a predetermined time period has elapsed, on the basis of the measurement values of the physical quantities measured by the environmental measurement devices 300 and the physical quantity setting values set in the environmental control devices 200.

In this case, the time at which the predetermined time period has elapsed is the time after a predetermined time period has elapsed from the time of measurement of the physical quantities that are provided to the physical quantity prediction model 171. Instead of the time of measurement of the physical quantities that are provided to the physical quantity prediction model 171, the time at which the arousal level control apparatus 100 (the communication unit 110) receives the measurement values of the physical quantities may be used.

In this case, the predetermined time period may be fixed at a constant time period, or may be made variable as a model parameter. The model parameter mentioned here is a set parameter in the physical quantity prediction model 171. The value of a model parameter will be referred to as a model parameter value.

The sub-models 172 and the arousal level prediction model 173 all take, as inputs, a physical quantity in a space in which a subject is located (the surrounding environment around the subject), and output a predicted value of an arousal level. Specifically, the sub-models 172 and the arousal level prediction model 173 are all mathematical models for computing a predicted value of an arousal level on the basis of the predicted value of the physical quantity computed by the physical quantity prediction model 171 and a variation in the physical quantity.

The sub-models 172 and the arousal level prediction model 173 may compute a predicted value of the variation in an arousal level in addition to the predicted value of the arousal level or instead of the predicted value of the arousal level. In the first example embodiment and the third example embodiment that are described below, examples of cases in which the arousal level control apparatus 100 performs arousal level control by using an optimization problem for maximizing the predicted value of the variation in the arousal level will be explained. In the second example embodiment and the fourth example embodiment that are described below, examples of cases in which the arousal level control apparatus 100 performs arousal level control by using an optimization problem for maximizing the predicted value of the arousal level will be explained.

The sub-models 172 are linear models corresponding to bases for generating the arousal level prediction model 173. The arousal level prediction model 173 is generated by a convex combination of a sub-model group (the multiple sub-models).

The mixing ratio computation unit 187 computes mixing ratios, which are ratios with which the multiple sub-models 172 are to be mixed (combined), and the arousal level prediction model generation unit 188 mixes the multiple sub-models 172 in accordance with the mixing ratios to generate the arousal level prediction model 173.

The number of sub-models 172 stored in the storage unit 170 need only be plural, and there is no limit on the specific number of sub-models 172.

The first example embodiment to the fourth example embodiment will explain examples of cases in which the storage unit 170 stores a single arousal level prediction model 173 in which all subjects are condensed into a single virtual subject corresponding to the average of all subjects, rather than being separate for each subject.

The control unit 180 controls the units in the arousal level control apparatus 100 to perform various processes. The control unit 180 is realized by a central processing unit (CPU) provided in the arousal level control apparatus 100 loading a program from the storage unit 170 and executing the loaded program.

The monitoring control unit 181 communicates with the environmental control devices 200 via the communication unit 110. By communicating with the environmental control devices 200, the monitoring control unit 181 acquires the device setting values set in the environmental control devices 200. Additionally, by communicating with the environmental control devices 200, the monitoring control unit 181 updates the device setting values of the environmental control devices 200. For example, the monitoring control unit 181 communicates with the environmental control devices 200 at constant intervals, and saves the device setting values acquired by communication together with timestamps of the times of acquisition (the times of reception). Saving mentioned here refers, for example, to storing data in the storage unit 170.

The monitoring control unit 181 sets the device setting values computed by the setting value computation unit 184 in the environmental control devices 200.

The first acquisition unit 182 communicates with the environmental measurement devices 300 via the communication unit 110, and acquires measurement values of physical quantities measured by the environmental measurement devices 300. For example, the first acquisition unit 182 communicates with the environmental measurement devices 300 at constant intervals, and saves the measurement values of the physical quantities acquired by communication together with timestamps of the times of acquisition (the times of reception). These timestamps can be considered to indicate the times of measurement of the physical quantities by the environmental measurement devices 300.

The second acquisition unit 183 communicates with the arousal level estimation devices 400, and acquires an estimated value of the arousal level of a subject. For example, the second acquisition unit 183 communicates with the arousal level estimation devices 400 at constant intervals and saves the estimated values of the arousal level acquired by communication together with timestamps of the times of acquisition (the times of reception). These timestamps can be considered to indicate the times of estimation of the arousal level by the arousal level estimation devices 400.

The estimated value of the arousal level of the subject will also be referred to as an arousal level estimate value.

The setting value computation unit 184 computes device setting values for the environmental control devices 200 such as to increase the arousal level of the user. For example, the setting value computation unit 184 computes the device setting values at constant intervals. The setting value computation unit 184 acquires device setting values from the monitoring control unit 181, acquires the measurement values of the physical quantities from the first acquisition unit 182, acquires the arousal level estimate value from the second acquisition unit 183, and computes the device setting values on the basis thereof. The setting value computation unit 184 outputs the computed device setting values to the monitoring control unit 181. The monitoring control unit 181 sets the device setting values in the environmental control devices 200 by transmitting the device setting values acquired from the setting value computation unit 184 to the environmental control devices 200 via the communication unit 110.

The setting value computation unit 184 computes setting values for controlling the arousal level of the subject by solving (or approximately solving) an optimization problem under constraint conditions relating to the physical quantities using the physical quantity prediction model 171 and the arousal level prediction model 173. The setting value computation unit 184 computes the device setting values so as to increase the arousal level by solving (or approximately solving) the optimization problem. Thus, the process by which the setting value computation unit 184 solves the optimization problem is an example of a process by which the value of an objective function such as an arousal level is made higher (or lower, or closer to a target value). The setting value computation unit 184 may compute the device setting values for the case in which the arousal level is maximized by solving (or approximately solving) the optimization problem.

In the optimization problem solved by the setting value computation unit 184, the physical quantity prediction model 171 is used as a first constraint condition, the arousal level prediction model 173 is used as a second constraint condition, and the condition that the device setting values of the environmental control devices 200 must be within a predetermined range is used as a third constraint condition. The setting value computation unit 184 solves the optimization problem including these constraint conditions. The predetermined range of the device setting values mentioned here is an allowable range that is determined by the specifications of the environmental control devices 200.

Additionally, the objective function of the optimization problem solved by the setting value computation unit 184 is, for example, a function for computing the total sum or the average value of predicted values of variations in arousal levels of one or more subjects and in one or more time step intervals. The setting value computation unit 184 computes the device setting values by solving the optimization problem so as to make the value of the objective function larger. The setting value computation unit 184 may compute the device setting values for the case in which the objective function is maximized.

The optimization problem solved by the setting value computation unit 184 will be referred to as an arousal level optimization problem (an arousal level optimization model). The arousal level optimization problem is configured as a mathematical model.

The combination of the setting value computation unit 184 and the monitoring control unit 181 is an example of a device control unit (device control means). Specifically, the setting value computation unit 184 uses the arousal level prediction model 173 to compute the device setting values. The monitoring control unit 181 controls the environmental control devices 200 by setting the device setting values computed by the setting value computation unit 184 in the environmental control devices 200.

The physical quantity prediction model arithmetic unit 185 reads the physical quantity prediction model 171 from the storage unit 170 and executes the model. Therefore, the physical quantity prediction model arithmetic unit 185 uses the physical quantity prediction model 171 to execute prediction of physical quantities.

The arousal level prediction model arithmetic unit 186 reads the arousal level prediction model 173 from the storage unit 170 and executes the model. Therefore, the arousal level prediction model arithmetic unit 186 uses the arousal level prediction model 173 to execute prediction of an arousal level.

The mixing ratio computation unit 187 computes the mixing ratios respectively for the multiple sub-models 172 on the basis of characteristic data of the subject. The characteristic data mentioned here may be history data regarding physical quantities influencing the arousal level of the subject and an estimated value of the arousal level of the subject. A vector created with this history data will be referred to as a history vector.

The arousal level prediction model generation unit 188 generates the arousal level prediction model 173 relating to the subject on the basis of these mixing ratios and the sub-models 172. Specifically, the arousal level prediction model generation unit 188 generates the arousal level prediction model 173 by computing a weighted average of the multiple sub-models 172, with the mixing ratios used for weighting factors.

There are multiple relationships between the arousal level of a person and physical quantities that influence the arousal level of the person, such as the room temperature, the variation in room temperature, the illuminance, and the variation in illuminance. These multiple relationships are each pre-stored in linear models in advance, and the storage unit 170 stores these linear models as the sub-models 172. The sub-models 172 are obtained by analyzing correlations between the physical quantities and the arousal levels of multiple test subjects such as, for example, 1000 people, classifying the obtained correlations into multiple classes, and linearly approximating the correlations between the physical quantities and the arousal levels in each class. The test subjects when generating the sub-models 172 may be people other than the subjects of the arousal level control by the arousal level control system 1.

The mixing ratio computation unit 187 computes the mixing ratios so as to obtain an arousal level prediction model 173 representing the relationship between the physical quantities and the arousal levels of the subjects on the basis of the physical quantities measured by the environmental measurement devices 300 and arousal level estimate values of the subjects estimated by the arousal level estimation devices 400. By generating the arousal level prediction model 173 on the basis of these mixing ratios, the arousal level prediction model generation unit 188 can obtain an arousal level prediction model 173 reflecting the characteristics of the subjects (individual differences and differences due to the psychosomatic state in the degree of influence that the surrounding environment has on the subjects of the arousal level control).

The mixing ratio computation unit 187 may compute mixing ratios for each subject, and the arousal level prediction model generation unit 188 may generate an arousal level prediction model 173 for each subject. In this case, the setting value computation unit 184 computes the device setting values of the environmental control devices 200 so as to maximize the total sum of the arousal levels of all subjects by, for example, solving an optimization problem for maximizing an average value obtained by averaging, across all subjects, the arousal levels computed for the subjects. The monitoring control unit 181 uses the device setting values computed by the setting value computation unit 184 to control the environmental control devices 200. As a result thereof, the total sum of the arousal levels for all subjects can be maximized.

On the other hand, the first example embodiment to the fourth example embodiment to be described below will explain examples of cases in which the mixing ratio computation unit 187 computes mixing ratios averaged across all subjects, and the arousal level prediction model generation unit 188 generates a single arousal level prediction model 173 in which all subjects are condensed into a single virtual subject corresponding to the average of all subjects, rather than being separate for each subject. In these cases, due to the linearity of the arousal level prediction model 173, the arousal level prediction model 173 becomes an arousal level prediction model 173 in which the arousal level prediction models 173 of all subjects are averaged. An arousal level prediction model obtained by averaging arousal level prediction models of multiple subjects in this way will be referred to as an averaged arousal level prediction model.

In the case that the arousal level prediction model generation unit 188 computes a single averaged arousal level prediction model (a single arousal level prediction model 173 in which all subjects are condensed into a single virtual subject corresponding to the average of all subjects) in this way, the setting value computation unit 184 solves an optimization problem for maximizing an arousal level in this averaged arousal level prediction model. As a result thereof, the setting value computation unit 184 computes the device setting values of the environmental control devices 200 so as to maximize the total sum of the arousal levels for all subjects in the same manner as in the case in which an arousal level prediction model 173 for each subject is used. The monitoring control unit 181 uses the device setting values computed by the setting value computation unit 184 to control the environmental control devices 200. As a result thereof, the total sum of the arousal levels for all subjects can be maximized in the same manner as in the case in which an arousal level prediction model 173 for each subject is used.

The display unit 120 displays the degree of influence of the physical quantities on increases and decreases in an arousal level for the sub-models 172. The display unit 120 also displays the mixing ratios for each subject computed by the mixing ratio computation unit 187.

By referring to the display on the display unit 120, the characteristics of a subject, for example, whether the arousal level of the subject is more easily influenced by the temperature or the illuminance, can be figured out. For example, in the case of operation by manually setting the air conditioning device and the lighting device without automatic control, the person who sets the devices may use settings such that the subject will not easily become drowsy by referring to the display on the display unit 120. Additionally, in the case in which the arousal level control apparatus 100 controls the environmental control devices 200, the effectiveness of arousal level control by the arousal level control apparatus 100 can be checked by referring to the display on the display unit 120.

An apparatus that displays the degree of influence of a physical quantity on increases and decreases in an arousal level for each sub-model 172 and that displays the mixing ratios for each subject in this way will be referred to as an arousal level characteristic display apparatus. The arousal level control apparatus 100 in FIG. 2 is an example of the arousal level characteristic display apparatus.

The arousal level characteristic display apparatus may not have the function of controlling the environmental control devices 200. For example, in the case of operation by manually setting the air conditioning device and the lighting device without automatic control as explained above, the arousal level characteristic display apparatus may be configured as a display-only device that does not control the environmental control devices 200.

Additionally, the functions of displaying the degree of influence of a physical quantity on increases and decreases in an arousal level and of displaying the mixing ratios for each subject are not essential to the arousal level control apparatus 100. For example, the arousal level control apparatus 100 may be configured so as not to be provided with the display unit 120.

### [Common Arousal Level Optimization Model]

Next, an example of an arousal level optimization model (an optimization problem) used by the setting value computation unit 184 to compute the device setting values will be explained. The setting value computation unit 184 computes the device setting values by performing mathematical optimization calculations on this arousal level optimization model.

This arousal level optimization model includes the constants, coefficients, variables, and functions indicated below.

### (Decision Variables)

Tₜ^{set}: Air conditioning temperature setting value at time step t
Lₜ^{set}: Lighting output setting value at time step t

The decision variables are variables with values computed by the setting value computation unit 184 in optimization operations. In the case of the example explained here, the setting value computation unit 184 computes the temperature set in an environmental control device 200 that is an air conditioning device and the illuminance set in an environmental control device 200 that is a lighting device by solving an optimization problem.

### (Dependent Variables)

A^{Δ}: Average value of predicted values of variations in arousal levels across subjects and time steps
Aᵢ^{Δ}: Average value of predicted values of variation in arousal level for subject i across time steps
A_{i,t}^{Δ}: Predicted value of variation in arousal level for subject i in time step t
Tₜ: Predicted value of temperature in time step t
Tₜ^{Δ}: Predicted value of temporal variation in temperature in time step t

It should be noted that the variation relative to one interval before time step t, i.e., the variation from time steps t−1 to t, is referred to as the variation in time step t. The temporal variation is the variation due to the passage of time (variation over time).

Lₜ: Predicted value of illuminance in time step t
Lₜ^{Δ}: Predicted value of temporal variation in illuminance in time step t

### (Constants and Coefficients)

T: Set of indices of time steps
N: Set of indices of subjects
T^{min}: Lower limit value of air conditioning temperature setting value
T^{max}: Upper limit value of air conditioning temperature setting value
L^{min}: Lower limit value of lighting output setting value
L^{max}: Upper limit value of lighting output setting value
Δτ: Time step width

### (Functions)

f_{A}: Arousal level variation prediction function (arousal level prediction model)
f_{T}: Temperature prediction function (one of physical quantity prediction models)
f_{L}: Illuminance prediction function (one of physical quantity prediction models)

### (Indices)

t: Index of time step
i: is Index of subject

The objective function of this arousal level optimization model is indicated by Expression (1).

$$\begin{matrix}[ Expression 1 ] & \\ {maximize}_{{T}_{t}^{set} , {L}_{t}^{set} , t ∈ 𝒯} {A}^{Δ} & ( 1 )\end{matrix}$$

A^{Δ} (average value of predicted values of variations in arousal levels across subjects and time steps) is indicated by Expression (2).

$$\begin{matrix}[ Expression 2 ] & \\ {A}^{Δ} = {mean}_{i ∈ N} {A}_{i}^{Δ} & ( 2 )\end{matrix}$$

Aᵢ^{Δ} (average value of predicted values of variation in arousal level for subject i across time steps) is indicated by Expression (3).

$$\begin{matrix}[ Expression 3 ] & \\ {A}_{i}^{Δ} = {mean}_{i ∈ 𝒯} {A}_{i , t}^{Δ} & ( 3 )\end{matrix}$$

A constraint condition that the device setting value of the air conditioning device among the environmental control devices 200 must be within a predetermined range is indicated by Expression (4).

[Expression 4]

T^{min}≤Tₜ^{set}≤T^{max} (4)

A constraint condition that the device setting value of the lighting device among the environmental control devices 200 must be within a predetermined range is indicated by Expression (5).

[Expression 5]

L^{min}≤Lₜ^{set}≤L^{max} (5)

A constraint condition for the physical quantity prediction model 171 relating to temperature is indicated by Expression (6).

[Expression 6]

Tₜ=f_{T}(Tₜ₋₁,Tₜ^{set}) (6)

A constraint condition for the physical quantity prediction model 171 relating to illuminance is indicated by Expression (7).

[Expression 7]

Lₜ=f_{L}(Lₜ₋₁,Lₜ^{set}) (7)

These constraint conditions for the physical quantity prediction models 171 indicate physical constraint conditions relating to the operation of the environmental control devices 200, such as the delay between when the device setting values are set in the environmental control devices 200 and when the physical quantities are actually reached to the device setting values.

Therefore, the explanatory variables (e.g., Tₜ₋₁ and Tₜ^{set} in Expression (6)) in the physical quantity prediction model 171 include parameters representing the physical quantities in a surrounding environment influencing the arousal level of a subject and parameters representing setting values of control devices influencing the physical quantities. Additionally, the explained variables (e.g., Tₜ in Expression (6)) in the physical quantity prediction model 171 include parameters representing predicted values of the physical quantities. Expression (6) and Expression (7) exemplify, by means of explicit functions, that predetermined processes that are indicated by the physical quantity prediction model 171 are applied to the values of the explanatory variables to compute the values of the explained variables. The constraint condition for the physical quantity prediction model 171 relating to temperature and the constraint condition for the physical quantity prediction model 171 relating to illuminance do not always need to be indicated by explicit functions as in Expression (6) and Expression (7).

An example of a constraint condition for the arousal level prediction model 173 is indicated by Expression (8).

[Expression 8]

A_{i,t}^{Δ}=f_{A}(Tₜ,Tₜ^{Δ},Lₜ,Lₜ^{Δ}) (8)

As indicated in Expression (8), the explanatory variables in the arousal level prediction model 173 include parameters representing physical quantities and parameters representing the temporal variations therein. Additionally, in the example in Expression (8), the explained variable in the arousal level prediction model 173 includes a parameter representing the predicted value of the temporal variation in the arousal level. Expression (8) exemplifies, by means of an explicit function, that a predetermined process that is indicated by the arousal level prediction model 173 is applied to the values of the explanatory variables to compute the value of the explained variable. It should be noted that the constraint condition for the arousal level prediction model 173 does not always need to be indicated by an explicit function as in Expression (8).

The arousal level indicated in Expression (8) has a large influence on the calculation time of the optimization problem in that the average value A^{Δ} computed by using Expression (2) and Expression (3) is used as the objective function in Expression (1). In particular, if Expression (8) is incorporated directly into the optimization problem, in other words, if Expression (8) is evaluated a number of times equal to the number of subjects, then the calculation time of the optimization problem will increase as the number of subjects increases. In this regard, scalability cannot be ensured in regard to the number of subjects.

The first example embodiment to the fourth example embodiment will explain examples of cases in which an arousal level prediction model averaged across all subjects is solved. By determining an average arousal level prediction model across all subjects before executing the optimization calculation, scalability can be obtained in regard to the number of subjects.

The constraint condition for the arousal level prediction model 173 indicates the manner of change in the arousal levels of the subjects in response to the physical quantities and changes therein.

Tₜ^{Δ} (predicted value of temporal variation in temperature in time step t) is indicated as in Expression (9).

[Expression 9]

Tₜ^{Δ}=|Tₜ−Tₜ₋₁| (9)

Lₜ^{Δ} (predicted value of temporal variation in illuminance in time step t) is indicated as in Expression (10).

[Expression 10]

Lₜ^{Δ}=|Lₜ−Lₜ₋₁| (10)

For example, the setting value computation unit 184 solves a mathematical programming problem for determining the values of the decision variables that maximize an objective function representing an average value of predicted values of temporal variations in arousal levels across all users and all time steps represented by Expressions (1) to (3) under the constraint conditions represented by Expressions (4) to (10). As a result thereof, the setting value computation unit 184 computes device setting values (the values of decision variable). The process executed by the setting value computation unit 184 can also, for example, be considered to be a process for computing setting values that maximize the value of the objective function under the constraint conditions using the arousal level optimization model as explained above. The process executed by the setting value computation unit 184 is not necessarily limited to being a process for computing setting values for the case in which the value of the objective function is maximized and, for example, may be a process for computing setting values for the case in which the value of the objective function is increased.

As explained above, Expressions (6) and (7) are constraint conditions regarding the physical quantity prediction model 171. Expressions (8) to (10) are constraint conditions regarding the arousal level prediction model 173. Expressions (4) and (5) are constraint conditions indicating that the device setting values of the environmental control devices 200 are within predetermined ranges.

The arousal level prediction model 173 is a mathematical model that can compute, with respect to time averages of physical quantities or temporal variations in physical quantities, a predicted value of the arousal level or the variation in the arousal level of a user when a predetermined time has elapsed. Arousal level prediction models in which the physical quantities are temperature and illuminance and the environmental control devices 200 corresponding to these physical quantities are respectively an air conditioning device and a lighting device are indicated, for example, by Expressions (8) to (10) explained above.

The calculation method for the arousal level optimization model is not limited to a specific method, and various known optimization calculation algorithms can be used.

The numerical values of the constants and coefficients will be explained.

The value of the time step width Δτ is set to an appropriate value, for example, within the range 15 to 30 minutes. From viewpoints such as the prediction accuracy and the arousal effects of the arousal level prediction model, the value of the time step width Δτ is preferably 15 minutes.

The set of indices of time steps T corresponds to the prediction horizon. In order to consider stimulation from environmental changes (such as hot and cold stimulation) due to temporal changes, there must be two or more time steps. For balance between the amount of calculation and the calculation time, there should preferably be three or four time steps.

The lower limit value T^{min} and the upper limit value T^{max} of the air conditioning temperature setting value may be set by a user by providing an input interface.

Similarly, the lower limit value L^{min} and the upper limit value L^{max} of the lighting output setting value may be set by a user by providing an input interface.

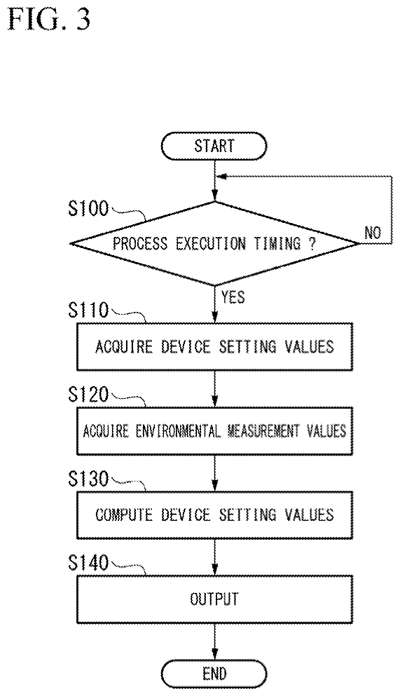

The calculations in the setting value computation unit 184 are executed by the procedure indicated in FIG. 3. The calculations are preferably executed at constant intervals of Δτ.

FIG. 3 is a flow chart indicating an example of the procedure for the setting value computation unit 184 to compute device setting values and to set the device setting values in the environmental control devices 200. FIG. 3 shows an example of the case in which the setting value computation unit 184 computes device setting values without using arousal level estimate values.

In the process in FIG. 3, the setting value computation unit 184 determines whether or not a timing for executing the process of computing device setting values has arrived (step S100). If it is determined that the execution timing has not arrived (step S100: No), then the process returns to step S100. As a result thereof, the setting value computation unit 184 waits until a timing for executing the process of computing the device setting values arrives.

In contrast, if it is determined that the timing for executing the process of computing the device setting values has arrived (step S100: Yes), then the setting value computation unit 184 acquires device setting values from the monitoring control unit 181 (step S110).

Additionally, the setting value computation unit 184 acquires environmental measurement values (measurement values of physical quantities measured by the environmental measurement devices 300) from the first acquisition unit 182 (step S120). Then, the setting value computation unit 184 computes device setting values (values for updating the device setting values in the environmental control devices 200) by solving the optimization problem as explained above (step S130). In step S130, the setting value computation unit 184 computes the device setting values without using arousal level estimate values.

The setting value computation unit 184 outputs the obtained device setting values to the monitoring control unit 181 (step S140). The monitoring control unit 181 transmits the device setting values obtained from the setting value computation unit 184 to the environmental control devices 200 via the communication unit 110, thereby setting the device setting values in the environmental control devices 200.

After step S140, the setting value computation unit 184 ends the process in FIG. 3.

### [Computation Method for Common Arousal Level Prediction Model]

Next, the arousal level prediction model will be explained. The arousal level control apparatus 100 uses an arousal level prediction model that reflects individual differences and differences due to the psychosomatic state in the degree of influence that the surrounding environment has on the subjects of arousal level control. As a result thereof, the arousal level control apparatus 100 reflects, in the arousal level control, individual differences and differences due to the psychosomatic state in the degree of influence that the surrounding environment has on the subjects of arousal level control.

The manner of changes in the arousal levels of subjects differs in accordance with individual differences and the psychosomatic states of the subjects. In order to obtain sufficient or desired arousal effects, it is preferable for arousal level control to reflect individual differences, and furthermore, it is preferably for arousal level control to reflect psychosomatic states.

As examples of individual differences in the arousal level, individual differences due to body weight or body fat percentage, and individual differences due to gender are known. For example, subjects who are high in body weight or in body fat percentage are known to have a tendency to have a smaller change in the arousal level in response to drops in environmental temperature than do subjects who are not high in body weight or in body fat percentage. Additionally, female subjects are known to have a tendency for the change in the arousal level due to the change in the environmental temperature to be larger than that for male subjects. Regarding the brightness of the environment also, there are known to be individual differences relating to sensitivity to light, more specifically relating to the level of inhibition of melatonin secretion due to light, depending on the subject.

Additionally, it is known that, even for the same subject, the manner in which an arousal level changes due to environmental changes differs depending on the psychosomatic state, such as whether the subject has had insufficient sleep, is fatigued, has recently eaten, is concentrating, or is distracted.

In order to handle such individual differences and differences in the psychosomatic state, for example, arousal level data for a subject himself/herself is analyzed and an arousal level prediction model for each subject is generated, thereby arousal level control can be made to reflect the characteristics of the subject. However, in order to construct an arousal level prediction model using only subject data, there is a need to comprehensively acquire arousal level data for the subject in advance for cases in which the surrounding environment is in various states. In other words, long-term data acquisition is required, and thus implementation is not easy.

Therefore, the storage unit 170 pre-stores multiple sub-models 172 that are not limited to use with specific subjects. Then, the arousal level prediction model generation unit 188 generates an arousal level prediction model 173 for a subject by combining these multiple sub-models 172 on the basis of subject data. As a result thereof, the arousal level control apparatus 100 can generate an arousal level prediction model 173 for the subject, and arousal level control can be made to reflect the characteristics of the subject, even when there is relatively little arousal level data for the subject.

Additionally, a model can be made to more accurately reflect the characteristics of a subject by using complicated non-linear functions to model the arousal level of the subject. However, in this case, there is a problem in that the amount of calculation for calculating the arousal level optimization model, i.e., for optimization calculation, becomes large. This problem relating to the amount of calculation can more specifically be divided into the following two problems.

First, during optimization calculation of the arousal level optimization model, complicated non-linear functions need to be repeatedly evaluated for each subject, thus increasing the amount of calculation as the number of subjects increases. In this way, there is a problem of a lack of scalability in regard to the number of subjects.

Additionally, there is a problem in that optimization calculation of complicated non-linear functions generally has a slow convergence speed to a global optimal solution, thus requiring long calculation times in order to obtain a satisfactory solution.

In contrast, in the arousal level control apparatus 100, the storage unit 170 stores linear sub-models 172. The arousal level prediction model generation unit 188 generates a linear arousal level prediction model 173 by combining the sub-models 172 on the basis of the mixing ratios computed by the mixing ratio computation unit 187. As a result thereof, in the arousal level control apparatus 100, the amount of calculation involved in the optimization calculation can made be relatively small, and the calculation time can be made relatively short.

Additionally, due to the arousal level prediction model 173 being linear, the arousal level prediction model generation unit 188 can generate an arousal level prediction model 173 that is common to multiple subjects and that is obtained by averaging the arousal level prediction models 173 of the multiple subjects. As a result thereof, the arousal level control apparatus 100 can ensure scalability in regard to the number of subjects.

In this way, according to the arousal level control apparatus 100, an arousal level prediction model 173 reflecting individual differences and differences due to the psychosomatic state in the manner of change in the arousal levels of subjects can be used to increase arousal effects, and optimization calculations used in prediction control can be efficiently performed with a relatively small amount of calculation. Additionally, according to the arousal level control apparatus 100, scalability can be ensured in terms of the amount of calculation in regard to the number of subjects.

Furthermore, in the arousal level control apparatus 100, the degree of influence of physical quantities on increases and decreases in an arousal level, which differs in accordance with a subject and/or the psychosomatic state thereof, can be computed as an intermediate parameter, and the degree of influence of a physical quantity on a change in the arousal level can be output and provided to subjects and managers. As a result thereof, subjects themselves can be informed of appropriate environments and managers can understand what types of characteristics are possessed by the subjects occupying a room, and this information can be used as a reference when manually setting air conditioning and/or lighting.

In the description of the arousal level prediction model, the variables, constants, coefficients, and functions below are used in addition to the variables, constants, coefficients, and functions explained above in the description of the arousal level optimization model.

### (Variables)

A: Average value of predicted values of arousal levels across subjects and time steps
Aᵢ: Average value of predicted values of arousal level for subject i across time steps
A_{*,t}: Average value of predicted values of arousal levels in time step t across subjects
A_{i,t}: Predicted value of arousal level for subject i in time step t
Uₜ: Vector representation of predicted values of physical quantities in time step t
Uₜ is a vector representation of the predicted values of the physical quantities (Tₜ, Tₜ^{Δ}, Lₜ, and Lₜ^{Δ}) for representing the arousal level optimization model in a matrix, as indicated in Expression (11).

[Expression 11]

Uₜ=[Tₜ,Tₜ^{Δ},Lₜ,Lₜ^{Δ},1]^{T} (11)

It should be noted that the superscript T in Expression (11) represents a transpose. As indicated in Expression (11), Uₜ is a vector (column vector) representing input elements that influence the arousal level of the subject, i.e., physical quantities in a surrounding environment around the subject, which are to be controlled. Uₜ includes predicted values of physical quantities (Tₜ, Tₜ^{Δ}, Lₜ, and Lₜ^{Δ}), and thus will be referred to as a physical quantity predicted value vector for time step t, or simply as a physical quantity prediction vector.

In Expression (11), the physical quantity predicted value vector Uₜ is defined as an extended input vector having the predicted values of the physical quantities (Tₜ, Tₜ^{Δ}, Lₜ, and Lₜ^{Δ}) and the constant 1 as elements. The extended input vector mentioned here is represented as a vector by adding the elements of the constant 1, which serves as identity elements, to the predicted values of the physical quantities that are the input elements influencing the arousal level of the subject.

Hereinafter, simple references to an extended input vector will mean the physical quantity predicted value vector Uₜ.

The physical quantity predicted value vector Uₜ is an example of an input to the sub-models 172 and an example of an input to the arousal level prediction model 173.

### (Constants and Coefficients)

wᵢ^{(s)}: Mixing ratio for sub-model s and for subject i

As explained below, “s” is an index of a sub-model, and is an identification number used for identifying each of the multiple sub-models 172. The sub-model 172 identified by index s is represented as sub-model s.

As explained above, the mixing ratios are ratios with which the multiple sub-models 172 are mixed. Here, the sub-models 172 are indicated by the input coefficients (or vector representations or matrix representations thereof) to be explained below. The arousal level prediction model generation unit 188 multiplies the mixing ratios by the input coefficients corresponding to the multiple sub-models 172, and adds the results obtained by multiplication to compute the arousal level prediction model 173.

wᵢ^{(s)} indicates the mixing ratio for each subject and for each sub-model 172.

As explained above, the mixing ratio computation unit 187 computes the mixing ratios on the basis of the physical quantities measured by the environmental measurement devices 300 and the arousal level estimate values of a subject estimated by the arousal level estimation devices 400, so as to obtain an arousal level prediction model 173 representing the relationship between the physical quantities and the arousal level of the subject.

The mixing ratio computation unit 187 may compute a mixing ratio wᵢ^{(s)} for each sub-model 172 and for each subject within the range from 0 to 1, as in Expression (12).

[Expression 12]

wᵢ^{(s)}∈[0,1] (12)

Alternatively, the mixing ratio computation unit 187 may compute a mixing ratio wᵢ^{(s)} for each sub-model 172 and for each subject as either 0 or 1, as in Expression (13).

[Expression 13]

wᵢ^{(s)}∈{0,1} (13)

wᵢ: Sub-model mixing ratio vector for subject i
wᵢ is a vector (column vector) collectively representing, for a single subject, the mixing ratios wᵢ^{(s)} for each subject and for the sub-models 172, as indicated in Expression (14).

[Expression 14]

wᵢ=[wᵢ⁽¹⁾, . . . ,wᵢ^{(M)}]^{T} (14)

As will be explained below, “M” is a positive integer constant indicating the number of sub-models 172.

The mixing ratio computation unit 187 may compute the values of the elements (the mixing ratios wᵢ^{(s)} for each subject and for the sub-models 172) in wᵢ so as to satisfy Expression (15).

[Expression 15]

∥wᵢ∥₁=1 (15)

∥wᵢ∥₁ represents the L1 norm (the sum of the absolute values of the elements in a vector) of wᵢ. Therefore, Expression (15) indicates that the total sum of the elements wᵢ^{(s)} of the sub-model mixing ratio vector wᵢ for subject i is 1. As a result thereof, multiplication by wᵢ is equivalent to computation of a weighted average.

By multiplying wᵢ by a collective representation of all M sub-models 172 in a single matrix (an input coefficient matrix θ to be described below) (i.e., by computing θwᵢ), an arousal level prediction model 173 for subject i (an input coefficient vector θᵢ for subject i to be described below) can be obtained by computing the weighted average of the sub-models 172.

wᵢ can also be represented as in Expression (16).

[Expression 16]

wᵢ=g(ϕᵢ) (16)

Expression (16) indicates that the sub-model mixing ratio vector wᵢ for subject i is computed from a sub-model mixing ratio output function g and a history vector ϕᵢ of subject i. As will be described below, the history vector ϕᵢ of subject i corresponds to history information indicating the correspondence relationship between the past arousal levels and the past physical quantities from time step t₀ to time step (t₀-t_{w}).

The sub-model mixing ratio output function g is determined by being learned in advance. The mixing ratio for each sub-model (a linear model represented by the input coefficient vector θ^{(s)}) is computed by the sub-model mixing ratio output function g.

The sub-model mixing ratio output function g may be a multi-class classifier. Specifically, the sub-model mixing ratio output function g can be realized with, for example, a multi-class support vector machine (SVM) or a neural network. In particular, if a neural network is to be used as the multi-class classifier, then a neural network having a network structure that can take chronological sequences into account, such as a recurrent neural network (RNN) or a long short term memory (LSTM), can be favorably used. The output from the multi-class classifier is preferably a probability that an input to the multi-class classifier belongs to a class, as in Expression (12) above. Alternatively, the output from the multi-class classifier may be a binary value indicating whether or not the input to the multi-class classifier belongs to a class, as in Expression (13) above.

w^{(s)}: Mixing ratio subject average value of sub-model s (a value obtained by averaging wᵢ^{(s)} (the mixing ratio for each subject and for each sub-model) across all subjects for one sub-model s)
w^{(s)} can be expressed as in Expression (17).

$$\begin{matrix}[ Expression 17 ] & \\ {w}^{( s )} = {mean}_{i ∈ N} {w}_{i}^{( s )} & ( 17 )\end{matrix}$$

w: Mixing ratio subject average vector (a collective vector representation, for all sub-models, of the mixing ratio subject average values w^{(s)} of the sub-models)
w can be expressed as in Expression (18).

[Expression 18]

w=[w⁽¹⁾, . . . ,w^{(M)}]^{T} (18)

w is equivalent to the value obtained by averaging wᵢ across all subjects. Since the L1 norm of wᵢ is 1, the L1 norm of w is also 1. Therefore, multiplication by w is also equivalent to computation of a weighted average.

As described above, the arousal level prediction model 173 for subject i is obtained by multiplying wᵢ by the input coefficient matrix θ (by computing θwᵢ). In contrast, an arousal level prediction model 173 (an input coefficient subject average vector θ_{avg} explained below) obtained by averaging the arousal level prediction models 173 across all subjects can be obtained by multiplying the mixing ratio subject average vector w by the input coefficient matrix θ (by computing θw).

Due to the linearity of the sub-models 172, the same values are obtained for the case in which an arousal level prediction model for each subject is generated using wᵢ, the arousal level for each subject is computed, and then the average value of the arousal levels across all subjects is computed, and the case in which an average arousal level prediction model across all subjects is generated using w and an arousal level is computed. When the setting value computation unit 184 computes an arousal level during the process of solving the above-mentioned optimization problem, even when there are many subjects, increases in the calculation time can be reduced by computing the average value of the arousal levels across all subjects using w (the mixing ratio subject average vector). In this respect, scalability can be obtained in regard to the number of subjects.

θⱼ^{(s)}: j-th input coefficient of sub-model s

The input coefficients are coefficients that are multiplied by predicted values of physical quantities in order to determine a predicted value of an arousal level or the variation in a predicted value of an arousal level, and that indicate the correlations between the physical quantities and an arousal level.

Here, as mentioned above, the physical quantity predicted value vector Uₜ is an example of an input to the sub-models 172. A vector collectively representing the input coefficients for the elements in this physical quantity predicted value vector Uₜ is an example of the sub-models 172. By computing the vector product thereof, the arousal level corresponding to the sub-models 172 can be computed.

θ^{(s)}: Input coefficient vector of sub-model s
θ^{(s)} is a collective vector representation of input coefficients for the predicted values of the physical quantities that are the elements in the physical quantity predicted value vector Uₜ, and can be expressed as in Expression (19).

[Expression 19]

θ^{(s)}=[θ₁^{(s)}, . . . ,θ_{S}^{(s)}]^{T} (19)

The elements θ₁^{(s)}, . . . , θ₅^{(s)} of the vector on the right side of Expression (19) indicate input coefficients that are multiplied respectively by the five elements of the physical quantity predicted value vector Uₜ. θ^{(s)} is an example of a sub-model 172.

θ: Input coefficient matrix

The input coefficient matrix θ is a collective vector representation of θ^{(s)} (the input coefficient vector of sub-model s) corresponding to each sub-model, and can be expressed as in Expression (20).

[Expression 20]

θ=[θ⁽¹⁾, . . . ,θ^{(M)}] (20)

M is a positive integer constant indicating the number of sub-models 172. The input coefficient matrix θ is an example in which all of the sub-models 172 are collectively expressed as a single matrix, and is used as a matrix that is common to all subjects. The numerical values of all elements in the input coefficient matrix θ are determined, for example, by being learned in advance.

θ_{avg}: Input coefficient subject average vector
θ_{avg} is expressed as in Expression (21).

[Expression 21]

θ_{avg}=θw (21)

Expression (21) corresponds to computing the input coefficient subject average vector θ_{avg}, corresponding to the average of input coefficient vectors of all subjects by computing the weighted average of the input coefficient vectors θ^{(s)} using the mixing ratio subject average values w^{(s)} of the sub-models s as weighting factors. As described above, the input coefficient subject average vector θ_{avg} is an example of the arousal level prediction model 173 obtained by averaging the arousal level prediction models 173 of all subjects. Therefore, the input coefficient subject average vector θ_{avg} is an example of the averaged arousal level prediction model.

θᵢ: Input coefficient vector for subject i

The input coefficient vector θᵢ for subject i is a vector indicating the degree of influence of the physical quantity predicted value vector Uₜ on the arousal level for subject i.

θᵢ can be expressed as in Expression (22).

[Expression 22]

θᵢ=θwᵢ (22)

Expression (22) corresponds to computing the input coefficient vector θᵢ for subject i by computing the weighted average of the input coefficient vectors θ^{(s)} using the mixing ratios wᵢ^{(s)} of the sub-models s as weighting factors. As described above, the input coefficient vector θᵢ for subject i is an example of the arousal level prediction model 173 for subject i.

ϕᵢ: History vector for subject i

The history vector for subject i is a vector having, as elements thereof, past arousal levels of subject i and the physical quantities at those times.

The history vector ϕᵢ for subject i is expressed as in Expression (23).

[Expression 23]

ϕᵢ=[A_{i,t}_{₀}, . . . ,A_{i,t}_{₀}₋ₜ_{_{w}},T_{i,t}_{₀}, . . . ,T_{i,t}_{₀}₋ₜ_{_{w}},L_{i,t}_{₀}, . . . ,L_{i,t}_{₀}₋ₜ_{_{w}}]^{T} (23)

The history vector ϕᵢ for subject i corresponds to history information representing the correspondence relationship between the past arousal levels and the past physical quantities from time step t₀ to time step (t₀-t_{w}).

The subscript i in the temperature term (T) in Expression (23) corresponds to the case in which different temperatures are to be used depending on the subject, for example, when there are multiple air conditioning devices. If a common temperature is to be used for all of the subjects, then this i is unneeded. Similarly, the subscript i in the brightness term (L) corresponds to the case in which different brightness values are to be used depending on the subject, for example, when there are multiple lighting devices. If a common brightness value is to be used for all of the subjects, then this i is unneeded.

M: Number of sub-models
W: Number of time steps
t₀: History origin time step
t_{w}: History time window size

The history origin time step to and the history time window size t_{w} indicate the time steps for which data is included in the history vector ϕᵢ. The data from the time step t₀ to the time step (t₀-t_{w}) is included in the history vector ϕᵢ.

γᵢ: Autoregressive coefficient for subject i

The autoregressive coefficient mentioned here is an autoregressive coefficient for an arousal level. If the explanatory variables in the arousal level prediction model 173 include an arousal level, then the arousal level prediction model 173 for subject i can be expressed as in Expression (24) using the autoregressive coefficient γᵢ for subject i.

[Expression 24]

A_{i,t+1}=γᵢA_{i,t}+θᵢ^{T}Uₜ₊₁ (24)

In Expression (24), when computing the arousal level A_{i,t+1} in time step t+1, the arousal level A_{i,t} in the previous time step (time step t) is used.

γ^{(s)}: Autoregressive coefficient of sub-model s
γ: Sub-model autoregressive coefficient vector

The sub-model autoregressive coefficient vector γ is expressed as in Expression (25).

[Expression 25]

γ=[γ⁽¹⁾, . . . ,γ^{(M)}]^{T} (25),

By using the sub-model autoregressive coefficient vector γ, the autoregressive coefficient γᵢ for subject i can be expressed as in Expression (26).

[Expression 26]

γᵢ=γ^{T}wᵢ (26)

Corrected initial arousal level for subject i The corrected initial arousal level Λᵢ for subject i can be expressed as in Expression (27).

[Expression 27]

Λᵢ=(γᵢ)^{W}A_{i,0} (27)

Λ: Corrected initial arousal level subject average

The corrected initial arousal level subject average A can be expressed as in

Expression (28).

$$\begin{matrix}[ Expression 28 ] & \\ Λ = {mean }_{i ∈ N} {Λ}_{i} & ( 28 )\end{matrix}$$

λ_{i,t}: Corrected input coefficient vector for subject i in time step t

The corrected input coefficient vector λ_{i,t} for subject i in time step t can be expressed as in Expression (29).

[Expression 29]

λ_{i,t}=(γᵢ)^{W-t}θᵢ (29)

λₜ: Corrected input coefficient subject average vector in time step t

The corrected input coefficient subject average vector λₜ in time step t can be expressed as in Expression (30).

$$\begin{matrix}[ Expression 30 ] & \\ {λ}_{t} = {mean}_{i ∈ N} {λ}_{i , t} & ( 30 )\end{matrix}$$

### (Functions)

g: Sub-model mixing ratio output function (vector function)
X^{T}: Transpose vector of vector X or transpose matrix of matrix X
∥x∥₁: L1 norm (the sum of the absolute values of elements in a vector) of vector x (index)
s: Index of sub-model (s=1, 2, . . . , M)
j: Index of input coefficient

### First Example Embodiment

The first example embodiment will explain an example of a case in which A^{Δ} (the average value of the predicted values of the variations in the arousal levels across subjects and time steps) is used as the objective function of the arousal level optimization model and an arousal level is not included as an explanatory variable in the arousal level prediction model.

In this case, the objective function of the arousal level optimization model can be expressed as in Expression (1) above.

When calculating the arousal level optimization model (i.e., when solving the optimization problem), the setting value computation unit 184 uses the input coefficient subject average vector θ_{avg} to determine A^{Δ}, which is to be maximized, by means of Expression (31).

$$\begin{matrix}[ Expression 31 ] & \\ {A}^{Δ} = {mean }_{i ∈ 𝒯} {θ}_{avg}^{T} {U}_{t} & ( 31 )\end{matrix}$$

The “θ_{avg}^{T}Uₜ” in Expression (31) can be rewritten as Expression (32) using Expression (11) and Expressions (18) to (21).

$$\begin{matrix} [ Expression 32 ] & \\ {θ}_{avg}^{T} {U}_{t} = {w}^{T} {θ}^{T} {U}_{t} = ( {w}^{( 1 )} {θ}_{1}^{( 1 )} + … + {w}^{( M )} {θ}_{1}^{( M )} ) {T}_{t} + ( {w}^{( 1 )} {θ}_{2}^{( 1 )} + … + {w}^{( M )} {θ}_{2}^{( M )} ) {T}_{t}^{Δ} + ( {w}^{( 1 )} {θ}_{3}^{( 1 )} + … + {w}^{( M )} {θ}_{3}^{( M )} ) {L}_{t} + ( {w}^{( 1 )} {θ}_{4}^{( 1 )} + … + {w}^{( M )} {θ}_{4}^{( M )} ) {L}_{t}^{Δ} + ( {w}^{( 1 )} {θ}_{5}^{( 1 )} + … + {w}^{( M )} {θ}_{5}^{( M )} ) & ( 32 )\end{matrix}$$

The variation in the arousal level can be computed by Expression (32), which is a linear regression expression, by using, as the values of the elements in θ, values reflecting the correlation between the physical quantities (the elements in Uₜ) and the variation in the arousal level. Therefore, θ_{avg} is an example of an arousal level prediction model. Each column in θ is an example of a sub-model and w is an example of a mixing ratio.

Here, as another method for computing A^{Δ}, the average of the variations in the arousal levels A_{i,t}^{Δ} across subjects i and time steps t may be computed for the subjects and the time steps. The variation in the arousal level A_{i,t}^{Δ} for subject i and time step t can be expressed as in Expression (33).

[Expression 33]

A_{i,t}^{Δ}=θᵢ^{T}Uₜ (33)

In this case, the variation in the arousal level A_{i,t}^{Δ} must be computed for all subjects using the arousal level prediction model (Expression (33)), and thus the amount of calculation increases as the number of subjects increases. In contrast, by using θ_{avg} as in Expression (31), the arousal level prediction model according to Expression (31) needs only be used, and there is no need to calculate other arousal level prediction models.

By the arousal level prediction model generation unit 188 calculating the input coefficient subject average vector θ_{avg} just once before performing the optimization calculation, there is no need for the arousal level prediction model (the input coefficient vector θᵢ for subject i) to be calculated for each subject in the optimization calculation. In the optimization calculation, the setting value computation unit 184 only needs to use θ_{avg} to compute the variation in the arousal level, and there is no need to calculate other arousal level prediction models. The setting value computation unit 184 basically only needs to compute the variation in the arousal level for one virtual subject corresponding to θ_{avg}, and the amount of calculation for the optimization calculation can be reduced to be substantially that for a single subject.

In this way, in the first example embodiment, by using θ_{avg}, which corresponds to the average of the arousal level prediction models of all subjects, control can be made to reflect differences in the arousal level due to individual differences and differences in the psychosomatic state, and the amount of calculation for the optimization calculation can be reduced to be substantially that for a single subject.

### Second Example Embodiment

The second example embodiment will explain an example of a case in which A (the average value of the predicted values of the arousal levels across subjects and time steps) is used as the objective function of the arousal level optimization model and an arousal level is not included as an explanatory variable in the arousal level prediction model.

In this case, the setting value computation unit 184 uses Expression (34) instead of Expression (1) above as the objective function of the arousal level optimization model.

$$\begin{matrix}[ Expression 34 ] & \\ {maximize}_{{T}_{t}^{set} , {L}_{t}^{set} , t ∈ 𝒯} A & ( 34 )\end{matrix}$$

Expression (34) differs from the case of Expression (1) in that what is maximized is the arousal level A rather than the variation in the arousal level A^{Δ}.

When calculating the arousal level optimization model, the setting value computation unit 184 determines A, which is to be maximized, by means of Expression (35), using the input coefficient subject average vector θ_{avg}.

$$\begin{matrix}[ Expression 35 ] & \\ A = {mean }_{i ∈ 𝒯} {θ}_{avg}^{T} {U}_{t} & ( 35 )\end{matrix}$$

The right side of Expression (35) is the same as the right side of Expression (31), and as in the case of the first example embodiment, the “θ_{avg}^{T}Uₜ” can be rewritten as Expression (32) above.

Regarding the fact that what is to be maximized is the arousal level A rather than the variation in the arousal level A^{Δ}, the change can be handled by setting different values of θ by means of learning. The arousal level can be computed by Expression (32), which is a linear regression expression, using, as the values of the elements in θ, values reflecting the correlations between the physical quantities (the elements in Uₜ) and the arousal level. In this case also, θ_{avg} is an example of an arousal level prediction model. Each element in θ is an example of a sub-model and w is an example of a mixing ratio.

Here, as another method for computing A, the average of the arousal levels A_{i,t} across subjects i and time steps t may be computed for the subjects and the time steps. The arousal level A_{i,t} for subject i and time step t can be expressed as in Expression (36).

[Expression 36]

A_{i,t}=θᵢ^{T}Uₜ (36)

In this case, the arousal level A_{i,t} must be computed for all subjects using the arousal level prediction model (Expression (36)), and thus the amount of calculation increases as the number of subjects increases. In contrast, by using θ_{avg} as in Expression (35), the arousal level prediction model according to Expression (35) needs only be used, and there is no need to calculate other arousal level prediction models.

Although the optimization calculation in the second example embodiment, when compared with the optimization calculation in the first example embodiment, differs in terms of whether the objective function is the variation in the arousal level A^{Δ} or the arousal level A, the operations that are performed are similar. Therefore, example advantageous effects similar to those in the case of the first example embodiment can also be obtained in the second example embodiment.

Specifically, by the arousal level prediction model generation unit 188 calculating the input coefficient subject average vector θ_{avg} just once before performing the optimization calculation, there is no need for the arousal level prediction model (the input coefficient vector θᵢ for subject i) to be calculated for each subject in the optimization calculation. In the optimization calculation, the setting value computation unit 184 only needs to use θ_{avg} to compute the arousal level, and there is no need to calculate other arousal level prediction models. The setting value computation unit 184 basically only needs to compute the arousal level for one virtual subject corresponding to θ_{avg}, and the amount of calculation for the optimization calculation can be reduced to be substantially that for a single subject.

In this way, in the second example embodiment, by using θ_{avg}, which corresponds to the average of the arousal level prediction models of all subjects, control can be made to reflect differences in the arousal level due to individual differences and differences in the psychosomatic state, and the amount of calculation for the optimization calculation can be reduced to be substantially that for a single subject.

### Third Example Embodiment

The third example embodiment will explain an example of a case in which AA (the average value of the predicted values of the variations in the arousal levels across subjects and time steps) is used as the objective function of the arousal level optimization model and an arousal level is included as an explanatory variable in the arousal level prediction model.

When the arousal level is included as an explanatory variable in the arousal level prediction model, the arousal level prediction model can be expressed as in Expression (37).

[Expression 37]

A_{i,t+1}=γᵢA_{i,t}+θᵢ^{T}Uₜ₊₁ (37)

When A^{Δ} (the average value of the predicted values of the variations in the arousal levels across subjects and time steps) is used as the objective function of the arousal level optimization model, the objective function of the arousal level optimization model can be expressed as in Expression (1) above. Expression (1) can be rewritten as in Expression (38).

$$\begin{matrix}[ Expression 38 ] & \\ {maximize}_{{T}_{t}^{set} , {L}_{t}^{set} , t ∈ 𝒯} {A}^{Δ} = {maximize}_{{T}_{t}^{set} , {L}_{t}^{set} , t ∈ 𝒯} { mean}_{i ∈ N} {mean}_{i ∈ 𝒯} ( {A}_{i , t} - {A}_{i , t - 1} ) & ( 38 )\end{matrix}$$

Expression (38) can be rewritten as in Expression (39).

$$\begin{matrix}[ Expression 39 ] & \\ {maximize}_{{U}_{1} , … {U}_{t}} {A}^{Δ} = {maximize}_{{T}_{t}^{set} , {L}_{t}^{set} , t ∈ 𝒯} ( {mean}_{i ∈ N} ( {A}_{i , W} - {A}_{i , 0} ) / W ) & ( 39 )\end{matrix}$$

Here, the set of indices of time steps T is specified by using the number of time steps W, as in Expression (40).

[Expression 40]

[symbol]

={1,2, . . . ,W} (40)

Expression (39) can be rewritten as in Expression (41).

$$\begin{matrix}[ Expression 41 ] & \\ {maximize}_{{U}_{1} , … {U}_{t}} {A}^{Δ} = {maximize}_{{T}_{t}^{set} , {L}_{t}^{set} , t ∈ 𝒯} ( {A}_{* , W} - {A}_{* , 0} ) / W & ( 41 )\end{matrix}$$

In Expression (41), “A_{*,0}” can be deemed to be a constant. As a result thereof, Expression (42) can be used as the objective function instead of Expression (41).

$$\begin{matrix}[ Expression 42 ] & \\ {maximize}_{{T}_{t}^{set} , {L}_{t}^{set} , t ∈ 𝒯} {A}_{* , W} & ( 42 )\end{matrix}$$

“A_{*,w}” in Expression (42) can be rewritten as in Expression (43).

[Expression 43]

A_{*,w}=Λ+λ₁^{T}U₁+λ₂^{T}U₂+ . . . +λ_{W}^{T}U_{W} (43)

The amount of calculation on the right side of Expression (43) does not depend on the number of subjects. As with the first example embodiment and the second example embodiment, even when multiple subjects in whom the arousal level characteristics differ due to personal differences or differences in the psychosomatic state are targets for arousal level control, by computing the corrected initial arousal level subject average A and the corrected input coefficient subject average vector λₜ just once before the optimization calculation, there is no need to use an arousal level prediction model for each of the subjects to determine the variation in the arousal level in the optimization calculation.

Additionally, Expression (43) indicates that it is sufficient to perform an optimization calculation for a single virtual subject corresponding to the average of the subjects, which corresponds to the corrected initial arousal level subject average Λ and the corrected input coefficient subject average vector λₜ. Therefore, the amount of calculation for the optimization calculation can be reduced to be substantially that for a single subject.

### Fourth Example Embodiment

The fourth example embodiment will explain an example of a case in which A (the average value of the predicted values of the arousal levels across subjects and time steps) is used as the objective function of the arousal level optimization model and an arousal level is included as an explanatory variable in the arousal level prediction model.

In this case, as in the case of the second example embodiment, the objective function of the arousal level optimization model can be expressed as in Expression (34) above. “A” in Expression (34) can be rewritten as in Expression (44).

[Expression 44]

A=Λ+λ₁^{T}U₁+λ₂^{T}U₂+ . . . +λ_{W}^{T}U_{W} (44)

Similarly to the right side of Expression (43) in the case of the third example embodiment, the right side of Expression (44) is a linear model that does not depend on the number of subjects. Therefore, the process in the fourth example embodiment is also similar to that for the case of the third example embodiment, and example advantageous effects similar to those for the case of the third example embodiment can be obtained.

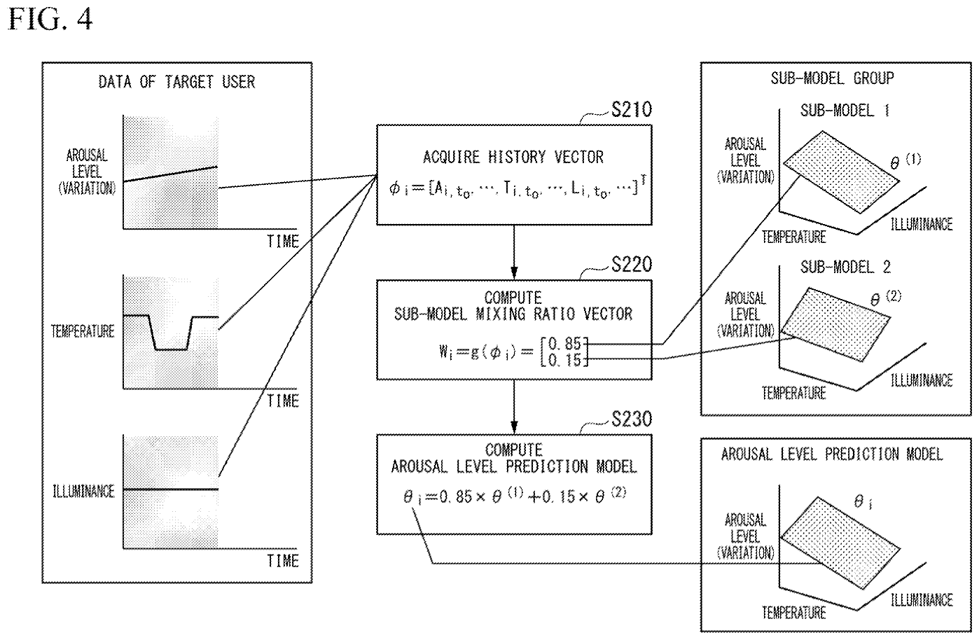

FIG. 4 is a diagram illustrating an example of a procedure for a process by which the arousal level control apparatus 100 generates an arousal level prediction model 173. FIG. 4 is common to the first example embodiment to the fourth example embodiment.

In the example in FIG. 4, there are two physical quantities, namely temperature and illuminance, and there are two sub-models 172.

In the process in FIG. 4, the setting value computation unit 184 acquires a history vector ϕᵢ, which is history information of past arousal levels and past physical quantities (step S210).

Next, the mixing ratio computation unit 187 inputs the acquired history vector ϕᵢ to a sub-model mixing ratio output function g to compute a sub-model mixing ratio vector wᵢ representing the degree to which each subject matches each sub-model (step S220). Here, the sub-models 172 are linear models having the physical quantities as explanatory variables, and the arousal level prediction model of the subject is combined as a convex combination of the sub-models.

Then, the arousal level prediction model generation unit 188 computes an arousal level prediction model (step S230). Specifically, a convex combination obtained by calculating a weighted average of the input coefficient vectors θ^{(s)} with the obtained sub-model mixing ratio vector wᵢ as weighting factors becomes an input coefficient vector θᵢ corresponding to the arousal level prediction model 173 of the subject.

After step S230, the arousal level control apparatus 100 ends the process in FIG. 4.

### Fifth Example Embodiment

The fifth example embodiment will explain a display of arousal level characteristics of subjects by the display unit 120. According to the fifth example embodiment, a manager and the subjects themselves can be provided with information regarding the arousal level characteristics of the subjects present in a room.

The display unit 120, for example, displays the input coefficient matrix θ and the sub-model mixing ratio vectors wᵢ. The input coefficient matrix θ is computed by means of learning in advance. The sub-model mixing ratio vectors wᵢ are computed by the mixing ratio computation unit 187.

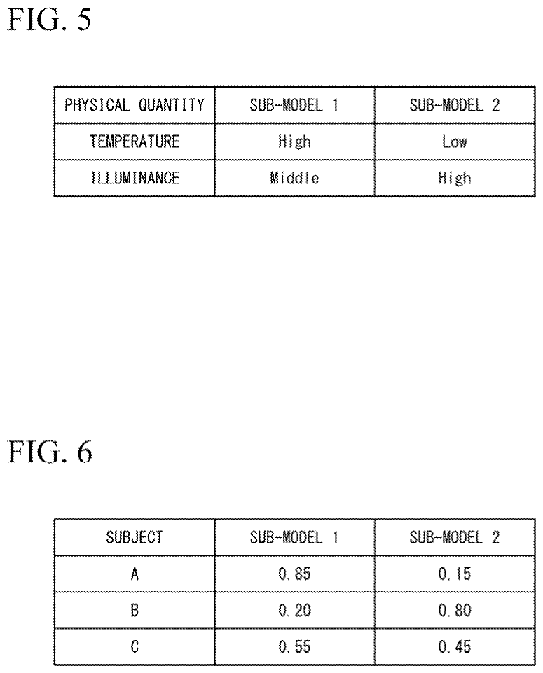

FIG. 5 is a diagram illustrating an example of a display of the input coefficient matrix θ by the display unit 120.

The input coefficient matrix θ indicates the degree of change in the arousal level in response to physical quantities in the surrounding environment for each sub-model. The display unit 120 indicates the input coefficient matrix θ in a tabular format. This table of the input coefficient matrix θ includes a “Physical quantity” column, a “Sub-model 1” column, and a “Sub-model 2” column. For the physical quantities of temperature and illuminance and for the sub-models, real number values indicating the degree of change in the arousal level are replaced by indications of a level, such as in the three stages “High”, “Middle”, and “Low”.

As a result thereof, a person (such as a manager or a subject) viewing the display is expected to be able to understand the degree of change in the arousal level more easily than in the case in which the display unit 120 displays the real number values directly. Alternatively, the display unit 120 may display the real number values directly.

It should be noted that the number of levels (the number of stages) displayed by the display unit 120 is not limited to the three stages as illustrated in FIG. 5 as long as there are multiple stages, and there may be two stages, or four or more stages. For example, the display unit 120 may replace real number values indicating the degree of change in the arousal level with the two levels “High” and “Low” for the display. Alternatively, the display unit 120 may replace real number values indicating the degree of change in the arousal level with N levels represented by level 1, level 2, . . . , level N (where N is an integer that satisfies N≥2) for the display.

FIG. 6 is a diagram illustrating an example of a display of sub-model mixing ratio vectors wᵢ by the display unit 120.

A sub-model mixing ratio vector wᵢ indicates the degree to which each of the sub-models 172 fits the arousal level characteristics of a subject. The display unit 120 indicates the sub-model mixing ratio vectors wᵢ in a tabular format. This table of the sub-model mixing ratio vectors wᵢ includes a “Subject” column, a “Sub-model 1” column, and a “Sub-model 2” column, and indicates the mixing ratios for sub-model 1 and sub-model 2, respectively, for each subject. The higher the mixing ratio, the more the sub-model can be considered to fit.

As in the case of the example in FIG. 5, the display unit 120 may replace real number values in the sub-model mixing ratio vectors wᵢ with indications of the level, such as in the three stages “High”, “Middle”, and “Low” for the display.

As in the case of the example in FIG. 5, the number of levels (the number of stages) displayed by the display unit 120 is not limited to the three stages as long as there are multiple stages, and there may be two stages, or four or more stages. For example, the display unit 120 may replace real number values indicating the sub-model mixing ratio vectors wᵢ with the two levels “High” and “Low” for the display. Alternatively, the display unit 120 may replace real number values indicating the sub-model mixing ratio vectors wᵢ with N levels represented by level 1, level 2, . . . , level N (where N is an integer that satisfies N≥2) for the display.

By the display unit 120 displaying the input coefficient matrix θ and the sub-model mixing ratio vectors wᵢ, people referring thereto can be notified of the arousal level characteristics of each subject. For example, in the sub-model mixing ratio vectors wᵢ in FIG. 6, subject A has a high mixing ratio for sub-model 1. Therefore, the arousal level characteristics of subject A can be considered to be arousal level characteristics that are close to those of sub-model 1, and the temperature can be figured out to have a large influence. Additionally, because subject B has a high mixing ratio for sub-model 2, subject B can be considered to have arousal level characteristics that are close to those of sub-model 2, and the illuminance can be figured out to have a large influence. As for subject C, because the mixing ratio for sub-model 1 and the mixing ratio for sub-model 2 are about the same, the influence of both temperature and illuminance can be figured out to be approximately medium. The display unit 120 may display not only the input coefficient matrix θ and the sub-model mixing ratio vectors but also other data such as the sub-model autoregressive coefficient vector γ.

As described above, the mixing ratio computation unit 187 computes the mixing ratio for each of multiple sub-models on the basis of characteristic data of subjects. The sub-models take, as inputs, physical quantities in a space in which the subjects are located (a surrounding environment around the subjects), and output predicted values of arousal levels. The arousal level prediction model generation unit 188 generates an arousal level prediction model 173 regarding the subjects on the basis of the mixing ratios and the sub-models. The monitoring control unit 181 and the setting value computation unit 184 use the arousal level prediction model 173 for controlling control target devices that influence the physical quantities.

According to the arousal level control apparatus 100, the arousal level prediction model can be made to reflect individual differences and differences due to the psychosomatic state in the degree to which physical quantities in the space in which the subjects are located (the surrounding environment around the subjects) influence the subjects of arousal level control. As a result thereof, according to the arousal level control apparatus 100, arousal level control can be made to reflect individual differences and differences due to the psychosomatic state in the degree to which physical quantities in the space in which the subjects are located (the surrounding environment around the subjects) influence the subjects of arousal level control.

Additionally, the arousal level control apparatus 100 uses sub-models that have been prepared in advance to generate an arousal level prediction model for a subject (an arousal level prediction model for each subject, or an arousal level prediction model averaged across the subjects). As a result thereof, the arousal level control apparatus 100 can generate an arousal level prediction model for the subject and perform arousal level control even in states in which there is relatively little subject data.

Additionally, the characteristic data is history data of physical quantities and estimated values of an arousal level.

As a result thereof, the arousal level control apparatus 100 can generate an arousal level prediction model by analyzing the correlation between the physical quantities and the arousal level. Additionally, the arousal level control apparatus 100 can perform arousal level control by using various physical quantities in accordance with the environment that is to be subjected to arousal level control.

Additionally, the arousal level prediction model generation unit 188 generates an arousal level prediction model 173 by computing the weighted average of multiple sub-models 172 with the mixing ratios as weighting factors.

As a result thereof, the arousal level prediction model generation unit 188 can generate an arousal level prediction model by linear combination with a relatively small amount of calculation; due to this feature, the load on the arousal level prediction model generation unit 188 is lightweight.

Additionally, the arousal level prediction model generation unit 188 generates an averaged arousal level prediction model obtained by averaging arousal level prediction models 173 of multiple subjects. The monitoring control unit 181 and the setting value computation unit 184 use the averaged arousal level prediction model to control the control target devices that influence the physical quantities.

As a result thereof, when performing an optimization calculation, the setting value computation unit 184 only needs to calculate an arousal level using the averaged arousal level prediction model, and there is no need to use an arousal level prediction model for each subject. Due to this feature, the arousal level control apparatus 100 can ensure scalability in regard to the number of subjects.

Additionally, the mixing ratio computation unit 187 computes mixing ratios for multiple sub-models on the basis of the characteristic data of subjects. The display unit 120 displays the degree of influence of physical quantities on increases and decreases in an arousal level for the sub-models, and displays the mixing ratios for the subjects.

As a result thereof, people referring to the display (e.g., a manager or the subjects) can figure out the arousal level characteristics of the subjects, and the arousal level control can be made to reflect the arousal level characteristics of the subjects.

Additionally, the characteristic data is history data of physical quantities and estimate values of an arousal level.

As a result thereof, the arousal level control apparatus 100 can generate an arousal level prediction model by analyzing the correlation between the physical quantities and the arousal level. Additionally, the arousal level control apparatus 100 can perform arousal level control by using various physical quantities in accordance with the environment that is to be subjected to arousal level control.

It should be noted that the sub-models 172 may be configured to be piecewise linear. For example, the sub-models 172 may be configured to be a combination of a linear portion (a partial model) for temperatures equal to or higher than a predetermined temperature, such as 20° C., and a linear portion for temperatures lower than the predetermined temperature. As a result thereof, more complicated models can be formed, and the example advantageous effects due to linearity can be obtained for each linear interval.

Alternatively, the sub-models may be configured to be linear models and the arousal level prediction model may be a rule-based model. For example, the arousal level prediction model may be obtained by combining the sub-models at different mixing ratios when the temperature is equal to or higher than a predetermined temperature, such as 20° C., and when the temperature is lower than the predetermined temperature. As a result thereof, more complicated models can be formed, and the example advantageous effects due to linearity can be obtained for each linear interval.

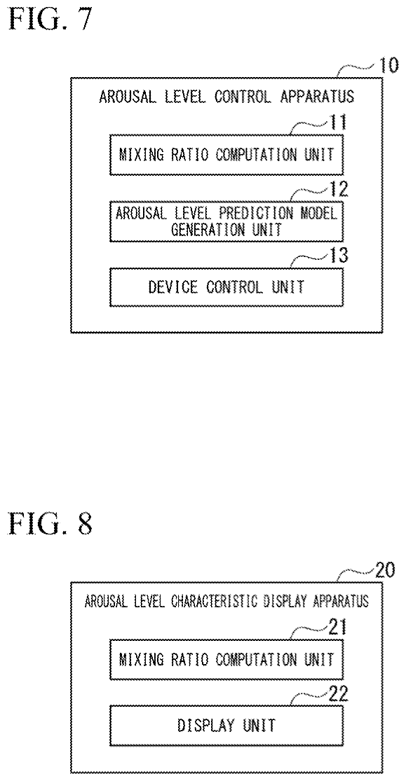

FIG. 7 is a diagram illustrating an example of a configuration of an arousal level control apparatus according to an example embodiment. The arousal level control apparatus 10 illustrated in FIG. 7 is provided with a mixing ratio computation unit 11, an arousal level prediction model generation unit 12, and a device control unit 13.

With this configuration, the mixing ratio computation unit 11 computes, on the basis of characteristic data of a subject, mixing ratios for multiple sub-models that take, as an input, a physical quantity in a space in which the subject is located and that output a predicted value of an arousal level. The arousal level prediction model generation unit 12 generates an arousal level prediction model relating to the subject on the basis of the mixing ratios and the sub-models. The device control unit 13 uses the arousal level prediction model for controlling a control target device that influences the physical quantity.

According to the arousal level control apparatus 10, the arousal level prediction model can be made to reflect individual differences and differences due to the psychosomatic state in the degree to which the physical quantity in the space in which the subject is located (the surrounding environment around the subject) influences the subject of arousal level control. As a result thereof, according to the arousal level control apparatus 10, arousal level control can be made to reflect individual differences and differences due to the psychosomatic state in the degree to which physical quantity in the space in which the subject is located (the surrounding environment around the subject) influences the subject of arousal level control.

Additionally, the arousal level control apparatus 10 uses sub-models that are prepared in advance to generate an arousal level prediction model for the subject (an arousal level prediction model for each of the subjects, or an arousal level prediction model averaged across the subjects). As a result thereof, the arousal level control apparatus 10 can generate an arousal level prediction model for the subject and perform arousal level control even in states in which there is relatively little subject data.

FIG. 8 is a diagram illustrating an example of a configuration of an arousal level characteristic display apparatus according to an example embodiment. The arousal level characteristic display apparatus 20 illustrated in FIG. 8 is provided with a mixing ratio computation unit 21 (mixing ratio computation means) and a display unit 22 (display means).

In this configuration, the mixing ratio computation unit 21 computes, on the basis of characteristic data of a subject, mixing ratios for multiple sub-models that take, as an input, a physical quantity in a space in which a subject is located and that output a predicted value of an arousal level. The display unit 22 displays the degree of influence of the physical quantity on increases and decreases in an arousal level for the sub-models, and displays the mixing ratios for each subject.

As a result thereof, people referring to the display (e.g., a manager or the subject) can figure out the arousal level characteristics of the subject, and the arousal level control can be made to reflect the arousal level characteristics of the subject.

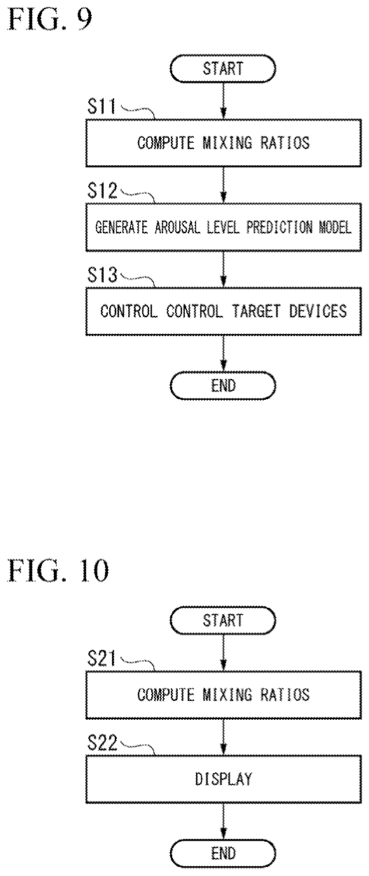

FIG. 9 is a diagram illustrating an example of a procedure for a process in an arousal level control method according to an example embodiment.

In the process in FIG. 9, mixing ratios for multiple sub-models that take, as an input, a physical quantity in a space in which a subject is located and that output a predicted value of an arousal level are computed on the basis of characteristic data of the subject (step S11), an arousal level prediction model for the subject is generated on the basis of the mixing ratios and the sub-models (step S12), and a control target device that influences the physical quantity is controlled using the arousal level prediction model (step S13).

According to this arousal level control method, the arousal level prediction model can be made to reflect individual differences and differences due to the psychosomatic state in the degree to which physical quantity in the space in which the subject is located (the surrounding environment around the subject) influences the subject of arousal level control. As a result thereof, arousal level control can be made to reflect individual differences and differences due to the psychosomatic state in the degree to which the physical quantity in the space in which the subject is located (the surrounding environment around the subject) influences the subject of arousal level control.

FIG. 10 is a diagram illustrating an example of a procedure for a process in an arousal level characteristic display method according to an example embodiment.

In the process in FIG. 10, mixing ratios for multiple sub-models that take, as an input, a physical quantity in a space in which a subject is located and that output a predicted value of an arousal level are computed on the basis of characteristic data of the subject (step S21), the degree of influence of the physical quantity on increases and decreases in an arousal level for each sub-model is displayed, and the mixing ratios for each subject are displayed (step S22).

As a result thereof, people referring to the display (e.g., a manager or the subject) can figure out the arousal level characteristics of the subject, and the arousal level control can be made to reflect the arousal level characteristics of the subject.

The configurations of the arousal level control apparatus 100, the arousal level control apparatus 10, and the arousal level characteristic display apparatus 20 are not limited to being configurations using computers. For example, the arousal level control apparatus 100 may be configured to use dedicated hardware, such as by being configured to use an application-specific integrated circuit (ASIC).

The present invention can realize arbitrary processes by making a central processing unit (CPU) execute a computer program.

In this case, the program may be stored by using various types of computer-readable media, for example, non-transitory computer-readable media, and supplied to a computer. Non-transitory computer-readable media include various types of tangible recording media. Examples of non-transitory computer-readable media include magnetic recording media (e.g., flexible disks, magnetic tape, and hard disk drives), magneto-optic recording media (e.g., magneto-optic discs), CD-read-only memory (ROMs), CD-Rs, CD-R/Ws, digital versatile discs (DVDs), Blu-ray (registered trademark) discs (BDs), and semiconductor memory (e.g., mask ROM, programmable ROM (PROM), erasable PROM (EPROM), flash ROM, and random access memory (RAM)).

While the present invention has been explained with reference to the example embodiments above, the present invention is not limited to the above-mentioned example embodiments. Various modifications that could be understood by a person skilled in the art can be made to the configuration and the specifics of the present invention within the scope of the present invention.

The present application claims the benefit of priority based on Japanese Patent Application No. 2019-075056, filed Apr. 10, 2019, the entire disclosure of which is incorporated herein by reference.

### INDUSTRIAL APPLICABILITY

The present invention is applicable, for example, to control of a physiological state of a subject. According to the present invention, physiological state control can be made to reflect at least one of individual differences and differences due to the psychosomatic state in the degree of influence that a physical quantity in a surrounding environment has on a subject of physiological state control.

### DESCRIPTION OF REFERENCE SIGNS

1 Arousal level control system
10, 100 Arousal level control apparatus
11, 21, 187 Mixing ratio computation unit
12, 188 Arousal level prediction model generation unit
13 Device control unit
20 Arousal level characteristic display apparatus
22 Display unit
110 Communication unit
120 Display unit
170 Storage unit
171 Physical quantity prediction model
172 Sub-model
173 Arousal level prediction model
180 Control unit
181 Monitoring control unit
182 First acquisition unit
183 Second acquisition unit
184 Setting value computation unit
185 Physical quantity prediction model arithmetic unit
186 Arousal level prediction model arithmetic unit
200 Environmental control device
300 Environmental measurement device
400 Arousal level estimation device

What is claimed is:

1. A physiological state control apparatus comprising:
a memory configured to store instructions; and
a processor configured to execute the instructions to:
compute, on the basis of characteristic data regarding a subject, mixing ratios for multiple sub-models that take, as an input, a physical quantity in a space in which the subject is located and that output a predicted value of a physiological index;
generate a physiological state prediction model for the subject on the basis of the mixing ratios and the sub-models; and
control a control target device that influences the physical quantity using the physiological state prediction model.

2. The physiological state control apparatus according to claim 1, wherein the characteristic data is history data for the physical quantity and an estimated value of the physiological index.

3. The physiological state control apparatus according to claim 1, wherein the processor is configured to execute the instructions to generate the physiological state prediction model by computing a weighted average of the multiple sub-models using the mixing ratios as weighting factors.

4. The physiological state control apparatus according to claim 1, wherein the processor is configured to execute the instructions to generate an averaged physiological state prediction model obtained by averaging physiological state prediction models for multiple subjects and control the control target device using the averaged physiological state prediction model.

5. A physiological state characteristic display apparatus comprising:
a memory configured to store instructions; and
a processor configured to execute the instructions to:
compute, on the basis of characteristic data regarding a subject, mixing ratios for multiple sub-models that take, as an input, a physical quantity in a space in which the subject is located and that output a predicted value of a physiological index; and
display a degree of influence of the physical quantity on increases and decreases in a physiological index value for the sub-models and display the mixing ratios for each subject.

6. The physiological state characteristic display apparatus according to claim 5, wherein the characteristic data is history data for the physical quantity and an estimated value of the physiological index.

7. A physiological state control method performed by a computer, the physiological state control method comprising:
computing, on the basis of characteristic data regarding a subject, mixing ratios for multiple sub-models that take, as an input, a physical quantity in a space in which the subject is located and that output a predicted value of a physiological index;
generating a physiological state prediction model for the subject on the basis of the mixing ratios and the sub-models; and
controlling a control target device that influences the physical quantity using the physiological state prediction model.

8.-10. (canceled)
